(19) United States
(12) Patent Application Publication
(10) Pub. No.: US 2016/0024460 A1
(43) Pub. Date: Jan. 28, 2016

(54) METHOD FOR CULTURING CELLS

(71) Applicant: Agency for Science, Technology and Research, Singapore (SG)

(72) Inventors: U Ming LIM, Singapore (SG); Lin Tang GOH, Singapore (SG); Say Kong NG, Singapore (SG)

(21) Appl. No.: 14/775,145

(22) PCT Filed: Mar. 12, 2014
(86) PCT No.: PCT/SG2014/000119

§ 371 Date: Sep. 11, 2015

(30) Foreign Application Priority Data
Mar. 12, 2013 (SG) 201301818-9

(51) Int. Cl. C12N 5/00

(52) U.S. Cl. CPC C12N 5/0037; C12N 2501/165; C12N 2501/12; C12N 2501/119

(57) ABSTRACT

A method of culturing a cell is provided comprising the step of contacting said cell with a cell culture medium comprising at least one of FGF, VEGF, or HGF. Also provided is a cell culture medium for culturing a cell comprising at least one of FGF, VEGF, or HGF; a kit for use in the method and a cell produced by the method.

### TECHNICAL FIELD

The present invention generally relates to in vitro cell culture. The present invention also relates to a cell culture medium and to a method of culturing cells using cell culture medium.

### BACKGROUND

Cell culture media provide the nutrients necessary to maintain and grow cells in a controlled, artificial and in vitro environment. Characteristics and compositions of the cell culture media vary depending on the particular cellular requirements. Important parameters include osmolarity, pH, and nutrient formulations.

Media formulations have been used to cultivate a number of cell types including animal, plant and bacterial cells. Cells cultivated in culture media catabolize available nutrients and produce useful biological substances such as monoclonal antibodies, hormones, growth factors and the like. Such products have therapeutic applications and, with the advent of recombinant DNA technology, cells can be engineered to produce large quantities of these products.

Cultured cells, for example, Chinese hamster ovary (CHO) cells are widely used host cell-lines for the production of recombinant bio-therapeutic proteins since they can perform post-translational modifications compatible with humans and can be scaled-up in suspension cultures. The development of a CHO production cell-line requires that the selected cell population derived from a single cell is homogenous to ensure that the protein produced is of consistent quality. This is achieved by dilution of an original cell population down to low cell densities and then growing them back to a larger cell population. The challenge with single cell growth is that mammalian cells grow slower or even do not survive when cultivated at low cell densities which can often lead to poor cloning efficiencies in the process of single cell cloning.

Success for this process necessitates a medium that supports cell growth at low cell densities. Fetal bovine serum (FBS) is typically supplemented to growth media to cultivate and expand single cells, primarily because it is rich in nutrients and growth factors. However, the use of the animal-derived FBS is now discouraged by regulators for a number of reasons including batch-to-batch variations, potential introduction of adventitious agents and re-adaptation of the cells from adherent to suspension growth in serum-free conditions. Other approaches include the use of conditioned media (CM) and plant hydrolysates, but these media are undefined and have batch to batch variations, which can result in inconsistent cloning efficiencies.

As such, efforts to develop a serum-free and fully defined single cell cloning media for CHO cells have resulted in the identification of growth stimulatory factors, such as insulin growth factor-1 (IGF-1), insulin, epidermal growth factor (EGF), basic fibroblast growth factor (bFGF) and albumin from different FBS lots. These growth factors have become important supplements to serum-free media formulations to improve the growth of CHO cells, although the cloning efficiencies achieved are still low.

While most of the CHO cell proteomic studies performed to date have focused on understanding the CHO intracellular proteome to improve cellular growth and productivity, only a few have looked at proteins in CHO cell culture medium. These autocrine growth factors are actively secreted by the cells themselves to promote cell growth in the exponential phase of cell culture when the growth rate is at its maximum. The fact that culture medium is commonly used in single cell cloning to improve the growth of CHO cells when cultivated at low cell density suggests that autocrine growth factors are secreted by the cells themselves for growth stimulation.

This, was also confirmed by other reports of autocrine growth factors in CM that promoted the growth of NSO and insect cell cultures. In these studies, fractions of concentrated CM stimulated cell proliferation when they were supplemented to the cultures, which indicated that autocrine proteins were involved in the regulation of cell growth. However, few autocrine growth factors in CM have been identified due to the low abundant nature of proteins secreted in culture supernatant. Hence, a highly sensitive proteomics method is needed to study secreted proteins.

Accordingly there is a need to provide a serum-free and fully defined single cell cloning media for CHO cells that demonstrates improved cloning efficiency that overcomes, or at least ameliorates, one or more of the disadvantages described above.

### SUMMARY

According to a first aspect, there is provided a method of culturing a cell comprising the step of contacting said cell with a cell culture medium comprising at least one of: a) FGF, b) VEGF, or c) HGF.

According to a second aspect, there is provided a cell culture medium for culturing a cell comprising at least one of FGF, VEGF, or HGF.

According to a third aspect, there is provided a kit when used in the method as described herein, comprising one or more containers of cell culture medium as claimed as described herein, together with instructions for use.

According to a fourth aspect, there is provided a use of a cell culture medium as described herein for culturing a cell.

According to a fifth aspect, there is provided a cell produced by the method of any one of claims as described herein.

### DEFINITIONS

The following words and terms used herein shall have the meaning indicated:

The term “ingredient” refers to any compound, whether of chemical or biological origin, that can be used in cell culture media to maintain the cell viability or promote the proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins, hydrolysates, serum and the like. Other ingredients that promote or maintain cultivation of cells ex vivo can be selected by those of skill in the art, in accordance with the particular need.

The term “cytokine” refers to a compound that induces a physiological response in a cell, such as growth, differentiation, senescence, apoptosis, cytotoxicity or antibody secretion. Included in this definition of “cytokine” are growth factors, interleukins, colony-stimulating factors, interferons and lymphokines.

By “cell culture” or “culture” is meant the maintenance of cells in an artificial, in vitro environment. However, it is to be understood that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture”.

By “cultivation” is meant the maintenance of cells in vitro under conditions favoring growth, differentiation or continued viability, in an active or quiescent state, of the cells. In this sense, “cultivation” may be used interchangeably with “cell culture” or any of its synonyms described above.

The phrases “cell culture, medium,” “culture medium” (plural “media” in each case) and “medium formulation” refer to a nutritive solution for cultivating cells and may be used interchangeably.

The term “contacting” refers to the placing of cells to be cultivated in vitro into a culture vessel with the medium in which the cells are to be cultivated. The term “contacting” encompasses mixing cells with medium, pipetting medium onto cells in a culture vessel, and submerging cells in culture medium.

By “culture vessel” is meant a glass, plastic or metal container that can provide an aseptic environment for culturing cells.

The term “combining” refers to the mixing or admixing of ingredients in a cell culture medium formulation.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−40 of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DISCLOSURE OF OPTIONAL EMBODIMENTS

Exemplary, non-limiting embodiments of a method of culturing a cell, will now be disclosed.

In one embodiment, there is provided a method of culturing cell comprising the step of contacting said cell with a cell culture medium comprising at least one of: a) FGF b) VEGF, or c) HGF.

In one embodiment the cell is a mammalian cell. The mammalian cell is an epithelial cell. In another embodiment the cell may be selected from a CHO, MDCK, HEK, HeLa, A549, COS, Hep-G2, hybridoma, NSO, BHK, Vero and PerC6, Age1.HN or MCF-7 cell. In yet another embodiment the cell is a CHO cell. The CHO cell may be a CHO-K1 cell, CHO-DukX-B11 cell or CHO-DG44 cell. In one embodiment the CHO cell is a single cell clone.

In one embodiment the culture medium comprises FGF, VEGF, and HGF. In the method described herein the culture medium comprises FGF-8, VEGF-c, and HGF. The FGF-8, may be provided at a concentration selected from about 0.02 ng/ml; from about 0.1 ng/ml to 250 ng/ml; about 0.1 ng/ml to 100 ng/ml; about 0.1 ng/ml to 50 ng/ml; about 0.1 ng/ml to 40 ng/ml; about 0.1 ng/ml to 30 ng/ml; about 0.1 ng/ml to 20 ng/ml; about 0.1 ng/ml to 10 ng/ml; about 0.1 ng/ml to 5 ng/ml; about 0.1 ng/ml to 4 ng/ml; about 0.1 ng/ml to 3 ng/ml; about 0.1 ng/ml to 2 ng/ml; about 0.1 ng/ml to 1 ng/ml; about 0.1 ng/ml to 0.5 ng/ml; about 0.1 ng/ml to 0.4 ng/ml; about 0.1 ng/ml to 0.3 ng/ml; about 0.1 ng/ml to 0.2 ng/ml.

In the method as described herein, the VEGF-c may be provided at a concentration selected from about 0.02 ng/ml; from about 0.02 ng/ml to 250 ng/ml; about 0.02 ng/ml to 100 ng/ml; about 0.02 ng/ml to 50 ng/ml; about 0.02 ng/ml to 40 ng/ml; about 0.02 ng/ml to 30 ng/ml; about 0.02 ng/ml to 20 ng/ml; about 0.02 ng/ml to 10 ng/ml; about 0.02 ng/ml to 5 ng/ml; about 0.02 ng/ml to 4 ng/ml; about 0.02 ng/ml to 3 ng/ml; about 0.02 ng/ml to 2 ng/ml; about 0.02 ng/ml to 1 ng/ml; about 0.02 ng/ml to 0.5 ng/ml; about 0.02 ng/ml to 0.4 ng/ml; about 0.02 ng/ml to 0.3 ng/ml; about 0.02 ng/ml to 0.2 ng/ml; about 0.02 ng/ml to 0.1 ng/ml; about 0.02 ng/ml to 0.05 ng/ml; about 0.02 ng/ml to 0.4 ng/ml; about 0.02 ng/ml to 0.03 ng/ml.

In the method as described herein, HGF may be provided at a concentration selected from about 0.02 ng/ml, from about 0.05 ng/ml to 250 ng/ml; about 0.05 ng/ml to 100 ng/ml; about 0.05 ng/ml to 50 ng/ml; about 0.05 ng/ml to 40 ng/ml; about 0.05 ng/ml to 30 ng/ml; about 0.05 ng/ml to 20 ng/ml; about 0.05 ng/ml to 10 ng/ml; about 0.05 ng/ml to 5 ng/ml; about 0.05 ng/ml to 0 ng/ml; about 0.05 ng/ml to 3 ng/ml; about 0.05 ng/ml to 2 ng/ml; about 0.05 ng/ml to 1 ng/ml; about 0.05 ng/ml to 0.5 ng/ml; about 0.05 ng/ml to 0.4 ng/ml; about 0.05 ng/ml to 0.3 ng/ml; about 0.05 ng/ml to 0.2 ng/ml; about 0.05 ng/ml to 0.1 ng/ml.

In one embodiment the FGF-8, VEGF-c and HGF are each provided at a concentration of about 10 ng/ml to 100 ng/ml. In another embodiment, the FGF-8 is at a concentration of about 100 ng/ml. In another embodiment, the VEGF-c is at a concentration of about 100 ng/ml. In another embodiment, the HGF is at a concentration of about 10 ng/ml. In yet another embodiment, the FGF-8 is hFGF-8. In yet another embodiment, the HGF is hHGF. In yet another embodiment, the VEGF-C is hVEGF-c. In yet another embodiment, FGF-8, VEGF-c and HGF are from other mammalian species, and are extracted from natural sources or produced recombinantly using bacterial, yeast, baculovirus or mammalian expression systems. It is to be appreciated that the optimal concentration of protein supplements to use will depend on the source of the protein supplement and can be determined by one of ordinary skill in the art using only routine experimentation.

In one embodiment, the culture medium may be a serum-free medium. In one embodiment, the culture medium is a serum-free medium supplemented with protein supplements. In another embodiment, the protein supplements are from other mammalian species, and are extracted from natural sources or produced recombinantly using bacterial, yeast, baculovirus or mammalian expression systems. In another embodiment, the protein supplements are IGF, insulin, EGF, albumin and HSA. In a preferred embodiment, the IGF is IGF-1. In yet another embodiment, the IGF-1 is recombinant human IGF-1. An example of a recombinant human IGF-1 is recombinant human IGF-1 from E-coli (Life Technologies; catalog number PHG0078). In another embodiment, the insulin is recombinant human insulin. An example of a recombinant human insulin is a recombinant human insulin from Saccharomyces cerevisiae (SAFC Biosciences; catalog number 91077C). In another embodiment, the EGF is recombinant human EGF. An example of a recombinant human EGF is a recombinant human EGF from E. coli (Life Technologies; catalog number PHG0311L). In another embodiment, the albumin is a recombinant human albumin. An example of a recombinant human albumin is a recombinant human albumin from Saccharomyces cerevisiae (Novozymes Biopharma; catalog number 200-010).

It is to be appreciated that the optimal concentration of protein supplements to use will depend on the source of the protein supplement and can be determined by one of ordinary skill in the art using only routine experimentation.

In one embodiment, the cell may be cultured for about 5 to about 60 days at about 33 to about 37° C. in about 5% to 8% CO₂. In another embodiment method as described herein results in an increase in the cloning efficiency of the cultured cell by about 10% to about 26% relative to a cloned cell cultured in a cell culture medium that is not supplemented with at least one of FGF, VEGF, or HGF in accordance with the method as described herein. In another embodiment method as described herein results in an increase in the cloning efficiency of the cultured cell by about 22% to about 44% relative to a cloned cell cultured in a cell culture medium that is not supplemented with at least one of FGF, VEGF, or HGF in accordance with the method as described herein.

In another embodiment there is provided a cell culture medium for culturing a cell comprising at least one of FGF, VEGF, or HGF. The cell culture medium as such may comprise: FGF-8, VEGF-c, and HGF.

In one embodiment the culture medium of the present invention is aqueous-based, comprising a number of ingredients in a solution of deionized, distilled water to form a serum free “basal media.” Any basal medium may be used in accordance with the disclosure. Examples of basal media include but are not limited to Dulbecco's Modified Eagle Medium (DMEM), Dulbecco's Modified Eagle's Medium/Nutrient Mixture F-12 (DMEM/F12), RPMI-1640, Basal Medium Eagle (BME), Minimal Essential Media (MEM), and Iscove's Modified Dulbecco's Medium (IMDM). In a preferred embodiment, the basal media is DMEM/F12. Ingredients which the basal media of the present disclosure may include are amino acids, lipids, fatty acids, hydrolysates from non-animal sources, vitamins, organic and/or inorganic salts, trace elements, buffering salts and sugars, and modified derivatives of such ingredients. In one embodiment, the serum free basal medium of the disclosure comprises one or more amino acids, one or more vitamins, one or more inorganic salts, adenine sulfate, ATP, one or more trace elements, deoxyribose, ethanolamine, D-glucose, glutathione, N-[2-hydroxyethyl]piperazine-N′-[2-ethanesulfonic acid] (HEPES) or one or more other zwitterion buffers, hypoxanthine, linoleic acid, lipoic acid, insulin, phenol red, phosphoethanolamine, putrescine, sodium pyruvate, thymidine, uracil and xanthine. Each of these ingredients may be obtained commercially, for example from Sigma (Saint Louis, Mo.).

Amino acid ingredients which may be included in the media of the disclosure include L-alanine, L-arginine, L-asparagine, L-aspartic acid, L-cystine, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. These amino acids may be obtained commercially, for example from Sigma (Saint Louis, Mo.).

Vitamin ingredients which may be included in the media of the disclosure include ascorbic acid magnesium salt, biotin, choline chloride, pantothenic acid, folic acid, i-inositol, menadione, niacinamide, nicotinic acid, paraaminobenzoic acid (PABA), pyridoxal, pyridoxine, riboflavin, thiamine-HCl, vitamin A acetate, vitamin B₁₂ and vitamin D₂. These vitamins may be obtained commercially, for example from Sigma (Saint Louis, Mo.).

Inorganic salt ingredients which may be used in the media of the present invention include CaCl₂, KCl, MgCl₂, MgSO₄, NaCl, NaHCO₃, Na₂HPO₄, NaH₂PO₄, and ferric citrate chelate or ferrous sulfate chelate. These inorganic salts may be obtained commercially, for example from Sigma (Saint Louis, Mo.).

Trace elements which may be used in the medium of the disclosure include irons of barium, bromium, cobalt, iodine, manganese, chromium, copper, nickel, selenium, vanadium, titanium, germanium, molybdenum, silicon, iron, fluorine, silver, rubidium, tin, zirconium, cadmium, zinc and aluminum.

Cytokines which may be used in the medium of the disclosure include growth factors such as epidermal growth factor (EGF), acidic fibroblast growth factor (aFGF), basic fibroblast growth factor (bFGF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), vascular endothelial cell growth factor (VEGF), transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon hematopoietic stem cells such as stem cell factor (SCF) and erythropoietin (Epo). These cytokines may be obtained commercially, for example from Life Technologies, Inc. (Rockville, Md.) or R&D Systems (Minneapolis, Minn.), and may be either natural or recombinant.

Additional ingredients that may be included in the present media are insulin and transferrin. Ferric citrate chelate or ferrous sulfate can be used in the present media as a substitute for transferrin. Additionally, recombinant insulin may be substituted for animal- or human-derived insulin.

The above ingredients, when admixed together in solution, form a “basal medium.” However, it will be appreciated that other basal media, can be equivalently used in accordance with the disclosure.

The medium ingredients can be dissolved in a liquid carrier or maintained in dry form. The osmolarity of the medium should also be adjusted to about 275-400 mOsm, preferably about 285-325 mOsm, and most preferably about 300-325 mOsm. The type of liquid carrier and the method used to dissolve the ingredients into solution vary and can be determined by one of ordinary skill in the art with no more than routine experimentation. Typically, the medium ingredients can be added in any order. The culture medium described herein may be sterilized to prevent unwanted contamination. Sterilization may be accomplished, for example, by filtration through a low protein-binding membrane filter of about 0.1-1.0 μm pore size (available commercially, for example, from Millipore, Bedford, Mass.) after admixing the ingredients to produce a sterile culture medium. Alternatively, subgroups of ingredients may be filter-sterilized and stored as sterile solutions. These sterile concentrates can then be mixed under aseptic conditions with a sterile diluent to produce a concentrated formulation. Autoclaving or other elevated temperature-based methods of sterilization are not favored, since many of the components of the present culture media are heat labile and will be irreversibly degraded by temperatures such as those achieved during most heat sterilization methods. As will be readily apparent to one of ordinary skill in the art, each of the components of the culture medium may react with one or more other components in the solution. Thus, the present disclosure encompasses the formulations as described above, as well as any reaction mixture which forms after the various ingredients are combined to form a final medium.

The optimization of the present media formulations was carried out using approaches described by Ham (Ham, R. G., Methods for Preparation of Media, Supplements and Substrata for Serum-Free Animal Culture, Alan R. Liss, Inc., New York, pp. 3-21 (1984)) and Waymouth (Waymouth, C., Methods for Preparation of Media, Supplements and Substrata for Serum-Free Animal Culture, Alan R. Liss, Inc., New York, pp. 23-68 (1984)). The optimal final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured.

Animal cells for culturing by the present invention may be obtained commercially, for example from ATCC (Rockville, Md.), Cell Systems, Inc. (Kirkland, Wash.) or Invitrogen Corporation (San Diego, Calif.). Alternatively, cells may be isolated directly from samples of animal tissue obtained via biopsy, autopsy, donation or other surgical or medical procedure.

The isolated cells can be plated according to the experimental conditions determined by the investigator. It is to be appreciated that the optimal plating and culture conditions for a given animal cell type can be determined by one of ordinary skill in the art using only routine experimentation. These cells can also be adapted to become attachment-independent suspension culture and to serum free culture media.

For routine culture conditions in accordance with the present disclosure, cells can be plated onto the surface of culture vessels without attachment factors. Alternatively, the vessels can be pre-coated with natural, recombinant or synthetic attachment factors or peptide fragments (e.g, collagen or fibronectin, or natural or synthetic fragments thereof). Isolated cells can also be seeded into or onto a natural or synthetic three-dimensional support matrix such as a preformed collagen gel or a synthetic biopolymeric material, or onto feeder layers of cells. Use of attachment factors or a support matrix with the medium of the present disclosure will enhance cultivation of many attachment-dependent cells in the absence of serum supplementation. Suspension cells can be cultivated in well-plates, T-flasks, roller bottles, cell factories, shake flasks, spinner flasks, wave bioreactors, stirred tank bioreactors, and other cell cultivation devices.

The cell seeding densities for each experimental condition can be optimized for the specific culture conditions being used. The cell culture media of the present invention may also be used to produce cell culture compositions comprising the present media and an animal cell. Animal cells preferably used in such compositions include, but are not limited to, cells obtained from mammals, birds (avian), insects or fish. Mammalian cells particularly suitable for use in such compositions include those of human origin, which may be primary cells derived from a tissue sample, diploid cell strains, transformed cells or established cell lines (e.g., HeLa), each of which may optionally be diseased or genetically altered. Other mammalian cells, such as hybridomas, CHO cells, COS cells, VERO cells, HeLa cells, 293 cells, PER-C6 cells, K562 cells, MOLT-4 cells, Ml cells, NS-1 cells, COS-7 cells, MDBK cells, MDCK cells, MRC-5 cells, WI-38 cells, SP2/0 cells, BHK cells (including BHK-21 cells) and derivatives thereof, are also suitable for use in forming the cell culture compositions of the present invention. Insect cells particularly suitable for use in forming such compositions include those derived from Spodoptera species (e.g., Sf9 or Sf21, derived from Spodoptera frugiperda) or Trichoplusa species (e.g., HIGH FIVE™ or MG1, derived from Trichoplusa spp). Tissues, organs, organ systems and organisms derived from animals or constructed in vitro or in vivo using methods routine in the art may similarly be used to form the cell culture compositions of the present invention. These cell culture compositions may be used in a variety of medical (including diagnostic and therapeutic), industrial, forensic and research applications requiring ready-to-use cultures of animal cells in serum-free media.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

In yet another embodiment there is provided a kit comprising one or more containers of cell culture medium as described herein, together with instructions for use.

In yet another embodiment there is provided use of a cell culture medium as described herein for culturing a cell.

In yet another embodiment there is provided a cell produced by the method as described herein. The cell may be used for bio-therapeutic production. In another embodiment the cell may be used for antibody production.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows a growth profile of CHO-K1 fed-batch bioreactor cultures in protein-free medium. Closed squares indicate viable cell counts and opened squares indicate percentage viability. Error bars represent the standard deviation calculated from data obtained from the experiment (n=2)

FIG. 2 shows the total number of proteins and peptides detected by LTQ Orbitrap Velos)(V° using the DDA method or/and SYNAPT G2 HDMS (SG2) using the MS^{E} method from day 2, 3, 4 & 5 CHO-K1 fed-batch supernatant.

FIG. 3. DDA and MS^{E} product ion spectra for, the Fibronectin (FN) tryptic peptide TGLDSPTGFDSSDITANSFTVHWVAPR. The spectra were generated during (a) DDA LC-MS/MS and (b), LC-MS^{E} analysis. Sequest searching of (a) was assigned to Fibronectin, whereas (b) did not return a match to any protein by Ion Accounting search algorithm.

FIG. 4. Cross-species database searches for LC-MS data processing (a) UniprotKb protein sequence alignment for Selenium binding protein-1 from Chinese hamster, human and mouse. The peptide detected (*) was matched against the human protein sequence but not the Chinese hamster or mouse. (b) Total number of proteins and peptides identified from combined LC-MS data matched against the UniprotKb Cricetulus griceus (Chinese hamster), Homo sapiens (human) and Mus musculus (mouse) database.

FIG. 5. Classification of CHO-K1 secreted proteins (a) Secretion prediction of the 290 secreted proteins. 163 secreted proteins had signal peptides, 106 were considered to be non-classically secreted and 121 were annotated as secreted/extracellular according to UniprotKb. (b) Sub-cellular location of proteins detected in the CHO-K1 secretome. Each of the 2512 identified proteins was assigned with cellular location. Cellular compartment assignment was performed using SignalP 4.0, SecretomeP 2.0 and gene ontology from UniprotKb. Out of the 2512 proteins, 11% were annotated as secreted. (c) Bar chart of the significantly enriched biological functions of proteins secreted by CHO-K1 cells. Identified secreted proteins were functionally annotated with GO-slim terms and statistically enriched GO functions were categorized into 39 main groups

FIG. 6. Western blot detection of secreted growth factors in concentrated Day 2 supernatant collected from CHO-K1 fed-batch bioreactor cultures. Recombinant human growth factors (rhGF) were used for the positive controls and Hela cell lysate was used for the negative controls.

FIG. 7. Network analysis of secreted growth factors identified from CHO-K1 cells by MetaCore mapping tool. The network was generated using the shortest path algorithm to map interaction between proteins. Nodes represent proteins; lines between the nodes indicate direct protein-protein interaction. The “*” denotes the identified secreted growth factors. The box indicated by “#” highlights identified membrane receptors corresponding to the 8 secreted growth factors.

FIG. 8. Western blot detection of membrane receptors corresponding to secreted growth factors identified from CHOK1 fed-batch bioreactor cultures. CHO-K1 membrane proteins were enriched using a sub-cellular fractionation kit. The membrane fraction was loaded on SDS-PAGE and probed with anti-receptor antibodies.

FIG. 9. The effect of LIF, FGF-8, HGF and VEGF-C on growth of CHO-K1 cells. CHO-K1 cells were seeded at 1×10⁴ cells/ml with the indicated concentrations of LIF, FGF-8, HGF and VEGF-C in 96 well plates for 72 hours and output was measured with the MTT assay. Error bars represent the standard deviation calculated from triplicate experiments (n=3) (*P<0.05; ** P<0.001).

FIG. 10. The effect of neutralizing antibodies on growth of CHO cells. (a) CHO-K1 cells were grown at a cell density of 1×10⁴ cells/ml in fresh media supplemented with indicated concentrations of neutralizing antibodies and isotype-specific control antibodies for 72 hours in 96 well plates. Cell growth was measured using the MTT cell proliferation assay. Values are indicated as the mean of triplicates ±standard deviation (*P<0.05; **P<0.001). (b) CHO-K1 cells were incubated with 10× concentrated Day 2 CM treated with neutralizing antibodies and isotype-specific control antibodies.

FIG. 11. The ERK signaling pathway cascade for FGF-8, HGF and VEGF-C.

FIG. 12. Western blot analyses: protein expression and phosphorylation of ERK1/2 in CHO-K1 cells cultured in protein-free media supplemented with 100 ng/ml FGF8, 10 ng/ml HGF and 100 ng/ml VEGF-C for up to 30 min.

FIG. 13. Analysis of single-cell cloning efficiency for CHO-K1 and CHO m250-9 cells. CHO cells were diluted to 1 cell/well and seeded into 96 well plates. Four conditions were tested which include basal media alone, basal media+100 ng/ml FGF-8+10 ng/ml HGF+100 ng/ml VEGF-C, basal media+Day2 CM and basal media+10% FBS. For each condition, a total of 30 replicates were used and wells with greater than 40% confluence at 28 days post seeding were scored as positive. The percentage of positive wells is indicated for both CHO-K1 and CHO m250-9 cell-lines. Error bars represent the standard deviation calculated from duplicate experiments (n=2) (*P<0.05).

FIG. 14. Analysis of single-cell cloning efficiency for CHO-DG44 cells. Basal media such as HyQ PF-CHO (HyQ) and CD-CHO™: HyQ (at 1:1) were supplemented with 100 ng/ml FGF-8+10 ng/ml HGF+100 ng/ml VEGF-C. For each condition, a total of 30 replicates were used and wells with greater than 40% confluence at 28 days post seeding were scored as positive. Error bars represent the standard deviation calculated from duplicate experiments (n=2) (*P<0.05).

FIG. 15. Performance of various cloning media for single cell growth of CHO-K1 and CHO-DG44 cells. CHO cells were diluted to 1 cell/well and seeded into 96 well plates. Basal media was used to culture CHO-K1 cells and CD-CHO™: HyQ (at 1:1) with HT for CHO-DG44 cells. Seven conditions were tested which included media alone; media+(A; 100 ng/ml FGF-8+10 ng/ml HGF+100 ng/ml VEGF-C); media+(B; 100 ng/ml IGF-1+10 μg/ml insulin+10 ng/ml EGF+2 g/1 human albumin); media+A+B; media+conditioned media (CM); media e+10% FBS; and media+10% FBS+A. For each condition, a total of 30 replicates were used and wells with greater than 40% confluence at 28 days post seeding were scored as positive. The percentage of positive wells is indicated for both CHO-K1 and CHO-DG44 cell-lines. Error bars represent the standard deviation calculated from duplicate experiments (n=2) (*P<0.05).

### EXAMPLES

Non-limiting examples of the invention, including the best mode, and a comparative example will be further described in greater detail by reference to specific Examples, which should not be construed as in any way limiting the scope of the invention.

### Example 1

### Effects of Conditioned Media (CM) on Low Cell Density Growth of CHO Cells

### Materials and Methods

### Supplementation of CM for CHO Cell Cultures

The CHO-K1 cell-line (CCL-61, ATCC) was adapted to suspension culture in a protein-free medium without components of animal origin. The cells were sub-cultured every 3 days in 125 mL Erlenmeyer shake-flasks (Corning) and incubated on a rotary shaker (110 rpm) in a humidified 5% CO2 environment at 37° C.

To investigate the effect of CM on low cell density cultures, CHO-K1 cells were seeded at inoculum sizes of 5×10⁵, 2.5×10⁵, 1×10⁵, 5×10⁴ and 1×10³ cells/ml in 20 mL batch shakeflask cultures. CM was harvested from separate day 2 CHO-K1 batch shakeflask cultures and concentrated by 10 fold with an Amicon® Ultra-15 centrifugal filter device (Merck, Millipore) fitted with low binding regenerated cellulose membrane (MWCO=10 kDa). CM was supplemented to each seeding density at 10% v/v, and cell density and viability were determined by counting in a haemocytometer using the trypan blue exclusion method. The initial growth rate, p, of the culture was calculated based on the exponential phase (0-72 hr), as described by the first-order differential equation:—

dt/dx=μx

where x denotes the viable cell density (VCD) and t denotes the time in culture.

### Results

Similar initial growth rates at inoculum sizes of 5×10⁵, 2.5×10⁵, 1×10⁵ and 5×10⁴ cells/ml were observed (Table 1), which suggested that cell growth was not affected at these seeding densities. In contrast, lower seeding densities of 1×10⁴ and 1×10³ cells/ml resulted in slower initial growth rates. The ability of CM to improve CHO cell growth at low seeding densities was investigated (Table 1). Upon addition of CM to CHO cell cultures seeded at 1×10⁴ cells/ml, initial growth rates improved from 0.033±0.002 hr⁻¹ to 0.051±0.002 hr⁻¹. This result confirms the potential of autocrine growth factors secreted in CHO cell CM to promote low cell density growth.

TABLE 1
Effects of CM on low cell density growth of CHO cells

| | Control | Conditioned media |
|---|---|---|
| Initial cell | (Initial growth rate, | (Initial growth rate, |
| density per ml | hr⁻¹) 0-72 hr | hr⁻¹) 0-72 hr |
| 5 × 10⁵ | 0.045 ± 0.003 | 0.045 ± 0.003 |
| 2.5 × 10⁵ | 0.046 ± 0.002 | 0.047 ± 0.002 |
| 1 × 10⁵ | 0.046 ± 0.002 | 0.050 ± 0.002 |
| 5 × 10⁴ | 0.044 ± 0.002 | 0.050 ± 0.002 |
| 1 × 10⁴ | 0.033 ± 0.002 | 0.051 ± 0.002 |
| 1 × 10³ | 0.010 ± 0.001 | 0.025 ± 0.002 |

### Example 2

### 2D LC-MS analysis of CM by Velos Orbitrap (DDA) and Synapt G2 (MS^{E})

### Materials and Methods

### Concentration of Proteins in Fed-Batch Culture Supernatant

To obtain supernatant samples, CHO-K1 fed-batch cultures were performed in two biological replicates using a protein-free medium. An initial working volume of 4.0 L of culture media was inoculated with a seeding density of 3.0×10⁵ cells/ml in a 5-L bioreactor (Sartorius). Dissolved oxygen concentration was maintained at 50% air saturation and culture pH was maintained at 6.9 using intermittent CO₂ addition to the gas mix and/or 7.5% (w/v) NaHCO₃ solution (Sigma-Aldrich).

Culture supernatant was collected from day 2, 3, 4 and 5 cultures. Collected samples were centrifuged (Sigma 3-16K) at 1000 rpm for 15 min to separate the cells from the supernatant. One tablet of Complete EDTA-free protease inhibitor cocktail (Roche) and 5 mM phenylmethylsulfonyl fluoride (Sigma-Aldrich) were added to the supernatant. The supernatant was passed through a 0.22 μm membrane filter (Merck Millipore) to remove residual cell debris and stored at −80° C. until further analysis. The methanol/chloroform (MCF) was used to concentrate proteins in all the samples for this study. Each 10-ml culture supernatant was mixed with 18 ml high purity water and 5 ml of chloroform/methanol (80:20 v/v, both from Merck). The mixture was centrifuged at 10,000 g, 15° C. for 30 min. The upper aqueous phase was discarded and 3 ml of methanol was further added to wash the precipitated protein pellet. The sample was then centrifuged at 10,000 g, 15° C. for 30 min, and the protein pellet was air dried after removal of the supernatant.

### Total Protein Assay and Trypsin Digestion

Each pellet-sample from MCF precipitation was dissolved in 50 μL of 8 M urea (Sigma Aldrich) with 0.1% (m/v) RapiGest™ SF surfactant (Waters Corporation). Total protein concentration was determined according to the Bradford method (Pierce), employing bovine serum albumin (Sigma) as a protein standard to generate the linear correlation plot. The measurements were performed in triplicate, at wavelength of 595 nm, using a Tecan Sunrise microplate reader (Tecan). 100 μg of protein was reduced with 25 mM Tris(2-carboxyethyl)phosphine solution (Pierce) for 15 min and carbamidomethylated with 55 mM iodoacetamide, at room temperature in the dark for 1 h. Proteolysis was performed with trypsin (Promega) at 1:50 (g/g) enzyme/protein ratio at 37° C. for 16 h. Post-digestion, each sample was dried in a SpeedVac Vacuum system (Savant Instruments) and resuspended in 200 μL of 0.1% (v/v) formic acid (FA). Prior to peptide separation by strong cation exchange (SCX) chromatography, each sample was centrifuged at 14,000 rpm for 10 min to remove any insoluble particulate.

### Strong Cation Exchange (SCX) Chromatography

The samples were diluted in the column equilibration solvent (10 mM monopotassium phosphate, 20% acetonitrile, pH 3.0) in up to 1 ml volume and injected onto a SCX column (PolyLC 2×150 mm, PolyMicro Technologies). Fractionation of the peptides was then performed by developing a gradient of potassium chloride (10 mM KH₂PO₄, 20% acetonitrile, 500 mM KCl, pH 3.0) over 60 min at a flow rate of 200 μl/min and collecting fractions every 2 min. For each sample, four fractions were collected for LC-MS analysis. Each of the 4 fractions was concentrated in a SpeedVac (Savant Instruments) and reconstituted in 50 μl of 1% formic acid, 2% methanol in Milli-Q water.

### LC-MS Configurations and Analysis

Two separate MS systems were used to acquire shotgun proteomics data from the processed CHO-K1 culture supernatant samples: LTQ Orbitrap Velos (Thermo Fisher Scientific) and SYNAPT G2 HDMS (Waters Corporation). Each mass spectrometer was coupled to a nanoAcquity UPLC system (Waters Corporation) fitted with identical trap (Symmetry C18, 5 μm, 180 μm×20 mm, Waters Corporation) and nano-analytical columns (BEH130 C18 1.7 μm, 75 μm×200 mm, Waters Corporation). For each sample run, the sample was trapped and desalted for 6 min, before applying the reversed phase elution profile. The elution program utilized two mobile phases, A (deionized water with 0.1% formic acid) and B (acetonitrile with 0.1% formic acid), where B was increased linearly from 2% to 40% over 180 min at flow rate of 300 nl/min. To minimize sample carryover, a dedicated column wash run (10% to 95% B in 30 min) followed by column re-equilibration (2% B for 30 min) were performed prior to the next sample injection.

Each MS system was set up and run with a method which enabled fast acquisitions of high quality peptide precursor and fragment ion data, with the desired precursor mass accuracy of ±5 ppm. For the LTQ Orbitrap Velos MS, the data-dependent MS/MS analytical workflow in positive ion mode was used. Each precursor survey scan (m/z: 300 to 1800) by the Orbitrap mass analyzer (resolution=60,000 FWHM) was linked to 10 MS/MS events using the 2D ion trap collision induced dissociation (CID) approach, with dynamic ion exclusion set at 60 s. This value was determined based on the observed mean peptide chromatographic peak width. All other instrument parameters were set up according to the manufacturer's suggested values for complex peptide samples. The nano-ESI source was fitted with a 30-μm stainless steel nano-bore emitter (Thermo Fisher Scientific) with 1.7 kV applied near the tip. On the SYNAPT G2 HDMS system, the positive ion MS^{E} acquisition method (cycle time 0.9 s) was employed to acquire the precursor and fragment ion data which eliminated the ion selection event of the quadrupole mass filter. The acquisition mass range for both low and high collision energy (CE) functions was 100 to 1950 in m/z scale with the TOF resolution setting of at least 10,000 FWHM. The profile for the high CE event was a linear ramp from 20 to 50 V in 0.9 s. During each LC-MS run, 100 fmol/μl Glu-fibrinopeptide B solution was delivered at 0.5 μl/min through the reference sprayer of the NanoLockSpray™ source (Waters Corporation) to provide real-time external lockmass correction. The lockmass channel was sampled at every 30 s. The sample sprayer was fitted with a 10-μm ID silica PicoTip emitter (New Objective) with 2.2 kV applied on the source stage.

### Results

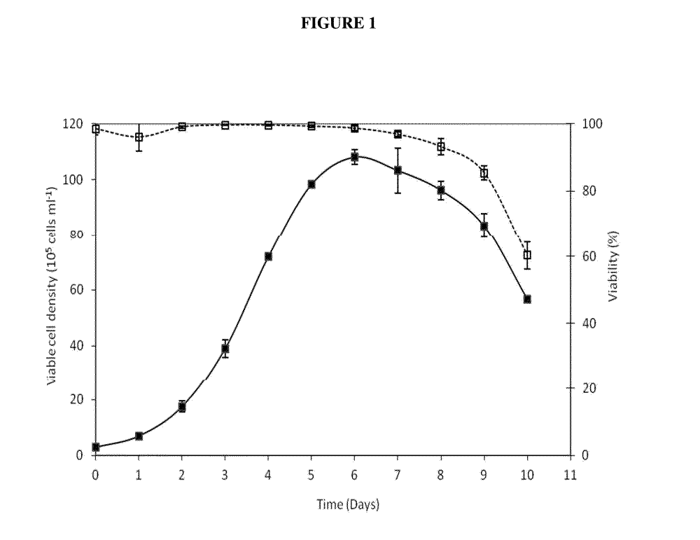

Suspension CHO-K1 cells were grown over a period of 10 days in two fed-batch bioreactor cultures and the average growth data is illustrated in FIG. 1. The culture reached a maximum viable cell density of 1.1×10⁷ cells/ml after 6 days and viabilities maintained above 95%. These CHO cells were previously adapted in protein-free media to avoid addition of fetal serum, which introduces between 50 and 600 mg of unspecified proteins and mask low abundant secreted proteins typically found in the μg range. To maximise the detection of low abundant secreted growth factors, CM were collected daily from day 2, 3, 4 & 5 so that cell density can expand significantly for more cells to produce growth factors. At higher cell densities, low abundant growth factors accumulate in the cultures which allow them to be detected at higher concentrations, assuming that they are not degraded by extracellular proteases or even consumed by the cells themselves. Upon sampling, proteins in CM were concentrated using an optimized MCF precipitation method and then reduced, alkylated and trypsinized to generate a peptide mixture for each of the samples. To simplify the complexity of the samples, the peptide mixture was then fractionated by SCX chromatography and fractions collected prior to LC-MS analysis.

For complimentary detection of low abundant secreted proteins in CM, the collected SCX fractions were acquired on both the LTQ Orbitrap Velos MS (VO) with the data dependent acquisition (DDA) method and the Synapt G2 HDMS (SG2) with the MS^{E} method. Combining the entire sample results, a total of 2512 proteins were identified from 24,359 peptide sequences with an empirical 1% FDR based on reversed sequence decoys and conservative criteria for protein assignment of at least 2 distinct peptides. It has been determined that the MCF precipitation method could manage higher protein recoveries of up to 86% (CV=1.4%).

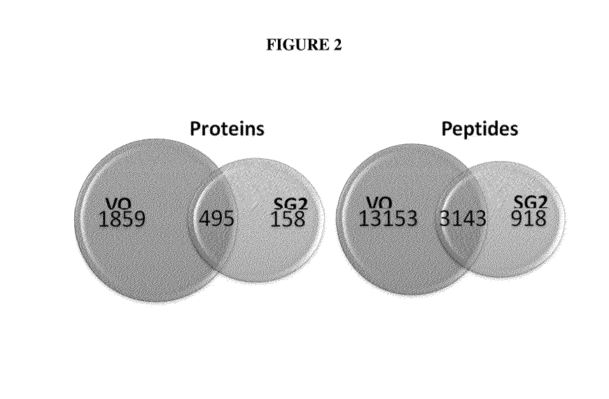

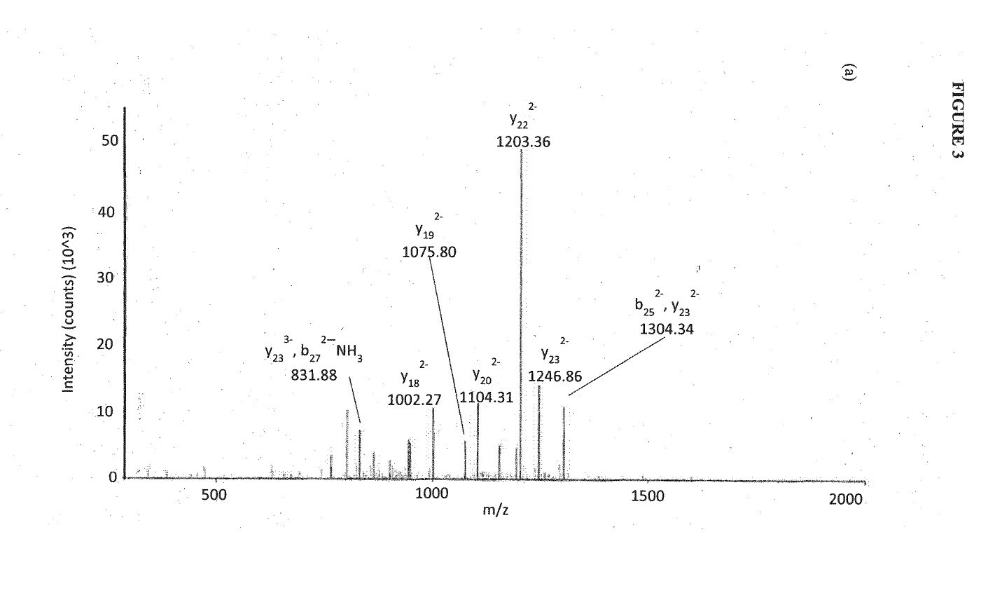

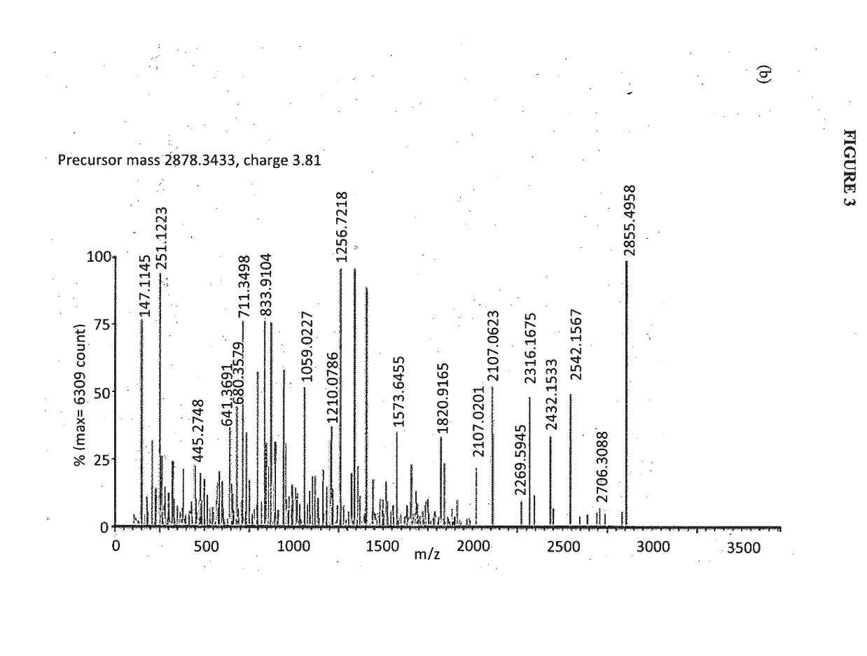

In general, the VO detected a higher number of protein IDs and peptides from day 2 to day 5 CM as opposed to SG2 (FIG. 2). It was observed that the lower numbers obtained from SG2 were mostly due to failure to generate good quality fragment ion spectra for peptides that are presented at low intensity levels. On the other hand, the VO allows these low intensity precursor ions to accumulate for a period of time, or until certain ion intensities are breached prior to selection for an MS/MS experiment, which explains the better quality fragment ion spectra. This behaviour is depicted in the generation of the product ion spectra for the Fibronectin peptide TGLDSPTGFDSSDITANSFTVHWVAPR (FIG. 3). The MS^{E} spectrum for this peptide did not return a match when searched against UniprotKb using Ion Accounting but was correctly assigned when the DDA spectrum was searched against UniprotKb using Sequest; a direct function of the difference between DDA and MS^{E} product ion generation.

Even though SG2 detected lower number of peptides, it still contributed proteins IDs and peptides unique to itself (FIG. 2). These lower intensity peptides could not be detected by the VO due to duty cycle limitations of MS/MS. Since MS/MS interrogation of individual precursor ions during DDA is conducted in a serial manner, the lower intensity peptides eluting at the same time with a large number of higher intensity precursors, were not interrogated at all or perhaps interrogated at a ‘suboptimal point’ in their chromatographic elution profile. In other words, these lower intensity peptides were mostly “skipped” at regions of high number of co-eluting peptides. As a result, detection of the missed peptides by SG2 led to the identification of additional secreted growth factors; for example, 2 peptides were uniquely identified by SG2 which matched to Growth regulated a protein (Table 2).

Other examples also include the peptide AVTEQGHELSNEER for 14-3-3 protein Pa which was detected by SG2 with a retention time at 50.09 min, but could not be detected by VO at a similar retention time. This result revealed that the peptide AVTEQGHELSNEER eluted at a time when the MS was performing CID on other higher intensity precursor ions, thus missing the opportunity of interrogating this peptide.

TABLE 2
Secreted growth factors identified by shotgun LC-MS in the supernatant of the CHO-K1 cell line

| | | | | Protein | | | | | Sample | | | |
|---|---|---|---|---|---|---|---|---|---|---|---|---|
| | | | Amino | Sequence | | | | | Identification^{f} | | | |
| | Protein | Gene | acid | Homology^{a} | MS | | | | Day | Day | Day | |
| Accession | Name | Name | length | (%) | Instrument^{b} | Peptides^{c} | SecretomeP^{d} | SignalP^{e} | 2 | 3 | 4 | [symbol] |
| G3ID16 | Brain-derived | BDNF | 187 | 94 | VO | 2.0 (0) | 0.83 | ✓ | (0) | (0) | (V) | [symbol] |
| | neurotrophic factor | | | | | | | | | | | |
| G3HXU8 | Fibroblast | FGF8 | 337 | 100 | VO, SG2 | 3.1 (1) | 0.63 | X | (B) | (B) | (V) | [symbol] |
| | growth factor 8 | | | | | | | | | | | |
| G3I473 | Growth regulated | CXCL1 | 107 | 71 | SG2 | 0.2 (0) | 0.82 | ✓ | (0) | (0) | (S) | [symbol] |
| | alpha protein | | | | | | | | | | | |
| G3HPQ5 | Hepatocyte | HGF | 728 | 89 | VO | 2.0 (0) | 0.44 | ✓ | (V) | (V) | (V) | [symbol] |
| | growth factor | | | | | | | | | | | |
| G3HG69 | Hepatoma-derived | HDGF | 205 | 88 | VO | 2.0 (0) | 0.44 | X | (V) | (V) | (V) | [symbol] |
| | growth factor | | | | | | | | | | | |
| G3I136 | Leukemia inhibitory | LIF | 158 | 74 | VO | 2.0 (0) | 0.45 | X | (V) | (V) | (V) | [symbol] |
| | factor | | | | | | | | | | | |
| G3IHR6 | Macrophage colony- | CSF1 | 552 | 74 | VO | 4.0 (0) | 0.38 | ✓ | (V) | (V) | (V) | [symbol] |
| | stimulating factor 1 | | | | | | | | | | | |
| G3HGL6 | Vascular endothelial | VEGFC | 346 | 72 | VO | 2.0 (0) | 0.62 | X | (V) | (V) | (V) | [symbol] |
| | growth factor C | | | | | | | | | | | |

^{a}UniprotKb protein sequence homology obtained from the human, mouse and Chinese hamster.
^{b}MS instrument used for LC-MS analysis; VO, LTQ Velos Orbitrap; SG2, SYNAPT G2 HDMS.
^{c}Peptides detected by VO, SG2 or common peptides (In parenthesis).
^{d}Secretion prediction according to SecretomeP server. Proteins with NN score ≧0.5 and without a predicted signal peptide (SignalP = X) are secreted by the non-classical secretory pathway.
^{e}Signal peptide prediction according to SignalP server. Proteins with predicted signal peptides (✓) are secreted by the classical secretory pathway.
^{f}MS detection of secreted growth factors from Day 2 to Day 5 samples of 2 biological replicates; VO (V), SG2 (S), both VO and SG2 (B), not detected (0). The colour represents the number of biological replicates the protein was detected; for example, yellow represents detection in 2 replicates, pink represents detection in 1 replicate and red represents not detected in any replicates. [symbol]
indicates data missing or illegible when filed

### Example 3

### Cross-Species Protein Identification

### Materials and Methods

### Data Processing and Protein Identification

LC-MS data from the LTQ Orbitrap Velos MS was processed using the Proteome Discoverer 1.3 software (Thermo Fisher Scientific) and those from Synapt G2 HDMS were processed using ProteinLynxGlobalSERVER™ v2.4 (Waters Corporation). On both software platforms, the LC-MS data were searched against the UniprotKb human (Homo sapiens), UniprotKb mouse (Mus musculus) and UniprotKb Chinese hamster (Cricetulus griceus) databases using the Sequest search engine for the LTQ Orbitrap Velos LC-MS data and the Ion Accounting search engine for the Synapt G2 HDMS LC-MS data. These search engines were set with the following possible protein modifications: 16 Da shift for oxidized Met and 6 and 8 Da shifts for stable isotope labeled Arg and Lys, respectively. Reverse database searching resulted in a specific false discovery rate (FDR) of 1% at the peptide and protein level. Proteins were identified by at least two unique peptides and trypsin endoprotease was selected for cleavage specificity allowing two missed cleavage sites. Redundant protein identifications from the results were consolidated based on protein gene name. Unreviewed Chinese hamster proteins were assigned with gene names from human and mouse homologs.

### Results

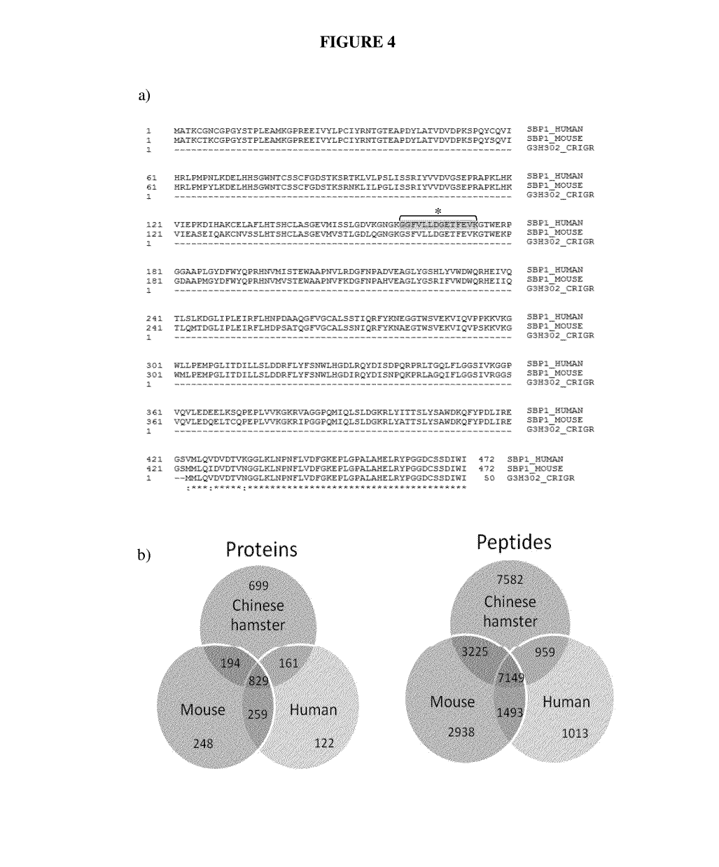

Although the estimated genomic size of the Chinese hamster is similar to other closely related organisms like the human and mouse, the size of the current Chinese hamster protein database is relatively small due to the lesser number of protein entries (SwissProt & TrEMBL) which currently stands at 24,433, a stark comparison to 249,044 and 84,127 for human and mouse respectively (Table 3). Out of the 24,433 protein entries, only 232 reviewed protein entries are non-redundant with manually curated experimental data. This means that there are many redundant protein entries for the unreviewed sections of the Chinese hamster protein database. In addition, many of the TrEMBL protein sequences translated from the recently published draft genome sequence of CHO-K134 are incomplete with missing regions, which would then result in unmatched LC-MS data. For example, a peptide GGFVLLDGETFEVK (*) for selenium-binding protein 1 was detected, which matched against the homologous protein sequence of the human but not against the Chinese hamster because of missing regions in the protein sequence (FIG. 4a). Consequently, searches of the LC-MS data against the Chinese hamster database alone would result in a high number of unmatched data and many redundant protein IDs that are difficult to filter away.

TABLE 3
Comparison of the estimated genome size and
UniprotKb protein database entries for human,
mouse and Chinese hamster (UniProt, 2012)

| | Estimated | Reviewed & | | |
|---|---|---|---|---|
| | Genome Size | Unreviewed | Reviewed | Unreviewed |
| | (Base pairs) | (UniprotKb) | (SwissProt) | (TrEMBL) |
| Human | 3.2 Gb | 249 044 | 25 991 | 223 053 |
| Mouse | 2.7 Gb | 84 127 | 16 967 | 67 960 |
| Chinese | 2.5 Gb | 24 433 | 232 | 24 201 |
| hamster | | | | |

To increase the number of protein IDs, searches against established databases of the human and mouse were performed. A major concern with database searches with surrogate organisms is whether the proteins identified are a true representation of proteins expressed by CHO cells. Based on our results, homology searches among the secreted growth factors that were identified from the UniprotKb human, mouse and Chinese hamster database, revealed that 70-100% of the protein sequences were identical. The high percentage of homology between human, mouse and Chinese hamster proteins validates the use of protein databases of closely related species to obtain cross-species protein identification. The significance of using alternate databases was evident with the finding that an additional 25% of protein IDs and 22% of peptides corresponding to 629 proteins and 5444 peptides were detected when the LC-MS data was searched against the human and mouse database separately (FIG. 4b).

These results demonstrated that LC-MS data searched against human and mouse databases enabled the detection of additional protein IDs to overcome the limitations of existing CHO protein database searches.

### Example 4

### Identification of Secreted Growth Factors

### Materials and Methods

### Bioinformatics Analysis

To identify secreted proteins, the cellular location of the proteins was determined as defined by UniprotKb. Secreted proteins were assigned based on cellular component classification of “secreted” and “extracellular”. All the proteins were analyzed for secretion pathways according to SecretomeP 2.0 Server (http://www.cbs.dtu.dk/services/SecretomeP/). If the neural network exceeded or was equal to a value of 0.50 (NN-score≧0.5), but no signal peptide was predicted, the protein is potentially secreted via a non-classical pathway. Those proteins with a predicted N-terminal signal sequence were confirmed using SignalP 4.0, available at http://www.cbs.dtu.dk/services/SignalP/. These proteins were considered to be secreted via a classical pathway. To determine the significant biological functions of secreted proteins, enriched functional groups of secreted proteins were annotated with Gene Ontology (GO) slim-terms using Gene Ontology (http://www.geneontology.org/). P-values for each GO-slim term were calculated using Fisher's exact test and a cutoff of 0.95 was used to determine biological significance.

### Results

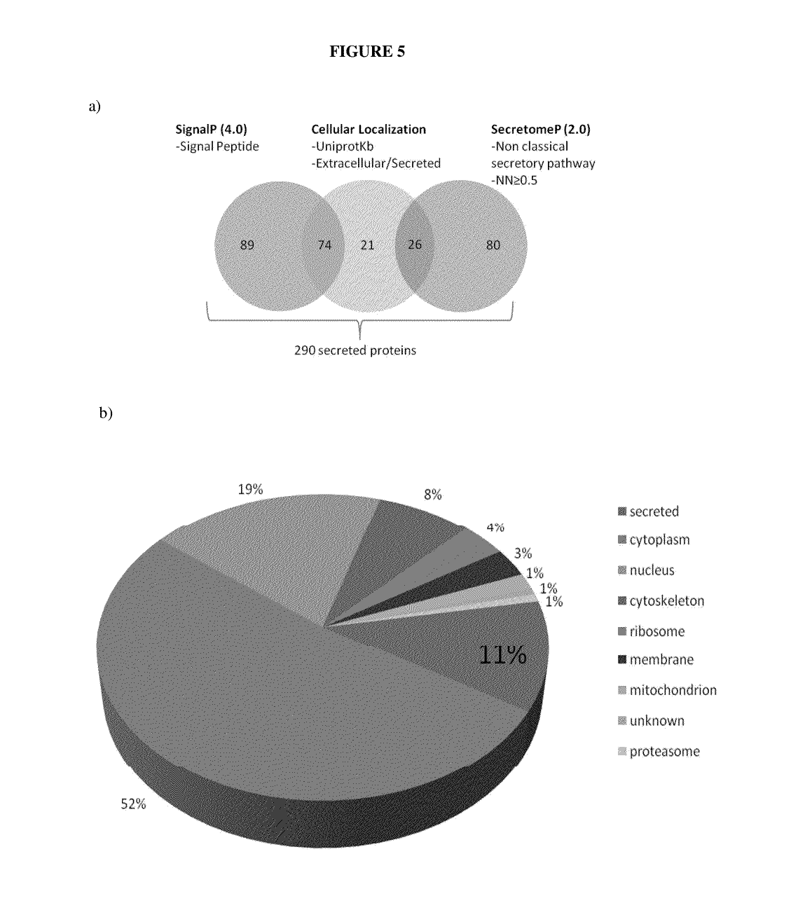

To classify secreted proteins, identified proteins were assigned with cellular location as defined by UniprotKb and checked using SignalP 4.0 and SecretomeP 2.0 to predict for secretion by classical or non-classical pathways. A total of 290 secreted proteins were identified; out of which 163 proteins had signal peptides, 103 proteins were predicted to be non-classically secreted and 121 proteins were annotated as ‘secreted’ or ‘extracellular’ (FIG. 5a). Secreted proteins constitute up to 110 of the proteins detected from CHO cell CM (FIG. 5b). A large number of host cell proteins predominantly from the cytoplasm, nucleus and cytoskeleton (2843 proteins) were also detected. Cell lysis due to cell death during harvest or mechanical damage by the bioreactor impellors may have contributed to the presence of these intracellular proteins in the final secretome repertoire.

Annotating secreted proteins with Gene Ontology (GO) slim-terms provided information on which functional groups were enriched for CHO secreted proteins (FIG. 5c) and p-values for each GO-slim term calculated using Fisher's exact test. Secreted proteins were identified to affect cell growth, apoptosis and product quality based on cell culture relevant functional groups like ‘regulation of cell proliferation’, ‘regulation of apoptotic process’, ‘regulation of cell death’, ‘N-glycan processing’, ‘proteolysis’ and ‘carbohydrate metabolism’ (Table 4). For the first time, 8 growth factors secreted by CHO-K1 cells have been identified from this list of identified protein targets (Table 2). In comparison, the UniprotKb database contains information for 92

TABLE 4
Secreted proteins affecting cell growth, apoptosis and product quality identified by shot-gun LC-MS in the supernatant of the CHO-K1 cell line

| | | | | | | |
|---|---|---|---|---|---|---|
| | | Gene | #Unique | | | GO Biological |
| Accession | Description | Name | Peptides | SecretomeP^{a} | SignalP^{b} | Process^{c} |

Cell Proliferation

| | | | | | | |
|---|---|---|---|---|---|---|
| Q99NV6 | Brain-derived neurotrophic factor | BDNF | 2 | 0.833 | | growth factor, anti-apoptosis |
| G3HXU8 | Fibroblast growth factor 8 | FGF8 | 3 | 0.632 | | growth factor, anti-apoptosis |
| G3I473 | Growth regulated alpha protein | CXCL1 | 2 | 0.819 | Signalp(1.0) | growth factor, cytokine/chemokine |
| G3HPQ5 | Hepatocyte growth factor | HGF | 2 | 0.438 | Signalp(1.0) | growth factor, anti-apoptosis |
| G3HG69 | Hepatoma-derived growth factor | HDGF | 2 | 0.437 | | growth factor |
| G3I136 | Leukemia inhibitory factor | LIF | 2 | 0.448 | | growth factor, cytokine/chemokine |
| G3IHR6 | Macrophage colony-stimulating factor 1 | CSF1 | 4 | 0.375 | Signalp(1.0) | growth factor, cytokine/chemokine |
| G3HGL6 | Vascular endothelial growth factor C | VEGFC | 2 | 0.623 | | growth factor |

Growth Inhibition

| | | | | | | |
|---|---|---|---|---|---|---|
| G3HJG6 | Decorin | DCN | 18 | 0.59 | Signalp(1.0) | growth inhibition, developmental |
| | | | | | | protein |
| G3IJ34 | Growth differentiation factor 8 | MSTN | 2 | 0.373 | Signalp(1.0) | growth inhibition |
| G3I5N6 | Insulin-like growth factor-binding | IGFBP4 | 2 | 0.841 | Signalp(1.0) | growth inhibition, developmental |
| | protein 4 | | | | | protein |
| G3IBH0 | Metalloproteinase inhibitor 1 | TIMP1 | 3 | 0.745 | Signalp(1.0) | growth inhibition, protease/protease |
| | | | | | | inhibitor |
| G3H3E6 | Metalloproteinase inhibitor 2 | TIMP2 | 3 | 0.78 | | growth inhibition, protease/protease |
| | | | | | | inhibitor |
| G3IKX2 | Pigment epithelium-derived factor | SERPINF1 | 2 | 0.359 | | growth inhibition, apoptosis |
| G3H584 | SPARC | SPARC | 2 | 0.929 | Signalp(1.0) | growth inhibition, apoptosis |
| G3HHV4 | Thrombospondin-1 | THBS1 | 3 | 0.43 | | growth inhibition, apoptosis, |
| | | | | | | cell adhesion |
| P04202 | Transforming growth factor β-1 | TGFB1 | 2 | 0.812 | Signalp(1.0) | growth inhibition, apoptosis |

Anti-Apoptosis

| | | | | | | |
|---|---|---|---|---|---|---|
| G3GZB2 | Acid ceramidase | ASAH1 | 3 | 0.792 | Signalp(1.0) | anti-apoptosis, lipid metabolism/ |
| | | | | | | transport |
| G3I5R0 | Angiopoietin-related protein 4 | ANGPTL4 | 2 | 0.673 | Signalp(1.0) | anti-apoptosis, developmental protein |
| Q99NV6 | Brain-derived neurotrophic factor | BDNF | 2 | 0.833 | | anti-apoptosis, growth factor |
| G3GTT2 | C-C motif chemokine 2 | CCL2 | 2 | 0.766 | Signalp(1.0) | anti-apoptosis, cytokine/chemokine |
| G3HNJ3 | Clusterin | CLU | 14 | 0.574 | Signalp(1.0) | anti-apoptosis, response to |
| | | | | | | oxidative stress |
| G3HXU8 | Fibroblast growth factor 8 | FGF8 | 2 | 0.632 | | anti-apoptosis, growth factor |
| G3HPQ5 | Hepatocyte growth factor | HGF | 2 | 0.438 | Signalp(1.0) | anti-apoptosis, growth factor |

Apoptosis

| | | | | | | |
|---|---|---|---|---|---|---|
| G3I4W7 | Cathepsin D | CTSD | 6 | 0.301 | Signalp(1.0) | apoptosis, protease/protease |
| | | | | | | inhibitor |
| G3HJG6 | Decorin | DCN | 18 | 0.59 | Signalp(1.0) | apoptosis, growth inhibition |
| P48538 | Galectin-1 | LGALS1 | 2 | 0.402 | | apoptosis |
| G3GZS9 | Interleukin-19 | IL19 | 2 | 0.767 | Signalp(1.0) | apoptosis |
| G3IKX2 | Pigment epithelium-derived factor | SERPINF1 | 2 | 0.359 | | apoptosis, growth inhibition |
| G3H584 | SPARC | SPARC | 2 | 0.929 | Signalp(1.0) | apoptosis, growth inhibition |
| G3HHV4 | Thrombospondin-1 | THBS1 | 3 | 0.43 | | apoptosis, growth inhibition, |
| | | | | | | cell adhesion |
| P04202 | Transforming growth factor β-1 | TGFB1 | 2 | 0.812 | Signalp(1.0) | apoptosis, growth inhibition |
| G3HCL3 | TNF ligand superfamily member 9 | TNFSF9 | 3 | 0.917 | | apoptosis |

Product Degradation

| | | | | | | |
|---|---|---|---|---|---|---|
| G3GUR0 | Calcium-dependent serine proteinase | PRCA | 13 | 0.76 | Signalp(1.0) | protease/protease inhibitor |
| G3H0L9 | Cathepsin B | CTSB | 3 | 0.799 | Signalp(1.0) | protease/protease inhibitor |
| G3I4W7 | Cathepsin D | CTSD | 6 | 0.301 | Signalp(1.0) | protease/protease inhibitor, |
| | | | | | | apoptosis |
| G3I4K2 | Cathepsin F | CTSF | 2 | 0.71 | Signalp(1.0) | protease/protease inhibitor |
| G3INC5 | Cathepsin L1 | CTSL1 | 2 | 0.505 | Signalp(1.0) | protease/protease inhibitor |
| Q9EPP7 | Cathepsin Z | CTSZ | 2 | 0.751 | Signalp(1.0) | protease/protease inhibitor |
| G3I1H5 | Legumain | LGMN | 9 | 0.714 | Signalp(1.0) | protease/protease inhibitor |
| G3GUV3 | Macrophage metalloelastase | MMP12 | 2 | 0.563 | Signalp(1.0) | protease/protease inhibitor |
| G3HRK9 | Matrix metalioproteinase-19 | MMP19 | 10 | 0.686 | Signalp(1.0) | protease/protease inhibitor |
| G3H8V1 | Matrix metalloproteinase-9 | MMP9 | 7 | 0.629 | Signalp(LO) | protease/protease inhibitor |
| G3IBH0 | Metalloproteinase inhibitor 1 | TIMP1 | 3 | 0.745 | Signalp(1.0) | protease/protease inhibitor, |
| | | | | | | growth inhibition |
| G3H3E6 | Metalloproteinase inhibitor 2 | TIMP2 | 3 | 0.78 | | protease/protease inhibitor, |
| | | | | | | growth inhibition |
| G3HA54 | Plasminogen activator inhibitor 1 | SERPINE1 | 2 | 0.62 | | protease/protease inhibitor |
| G3I2U1 | Serine protease 27 | PRSS27 | 2 | 0.749 | Signalp(1.0) | protease/protease inhibitor |
| G3HY12 | Stromelysin-3 | MMP11 | 2 | 0.57 | | protease/protease inhibitor |

Protein Glycosylation

| | | | | | | |
|---|---|---|---|---|---|---|
| G3H559 | Alpha-mannosidase 2 | MAN2A1 | 4 | 0.575 | Signalp(1.0) | carbohydrate metabolic process |
| G3IG18 | Alpha-mannosidase 2x | MAN2A2 | 2 | 0.575 | Signalp(1.0) | carbohydrate metabolic process |
| G3HLX3 | Alpha-N-acetylglucosaminidase | NAGLU | 5 | 0.656 | Signalp(1.0) | carbohydrate metabolic process |
| G3IDU7 | Beta-glucuronidase | GUSB | 2 | 0.553 | Signalp(1.0) | carbohydrate metabolic process |
| G3GWD3 | Epididymis-specific alpha-mannosidase | MAN2B2 | 3 | 0.527 | | carbohydrate metabolic process |
| G3HTE5 | Lysosomal alpha-glucosidase | GAA | 11 | 0.757 | Signalp(1.0) | carbohydrate metabolic process |
| G3HCV4 | Lysosomal alpha-mannosidase | MAN2B1 | 6 | 0.614 | Signalp(1.0) | carbohydrate metabolic process |
| G3I064 | Neutral alpha-glucosidase AB | GANAB | 8 | 0.649 | Signalp(1.0) | carbohydrate metabolic process |
| G3IIB1 | Sialate O-acetylesterase | SIAE | 2 | 0.753 | Signalp(1.0) | carbohydrate metabolic process |
| G3HZE3 | Sialidase-1 | NEU1 | 2 | 0.758 | | carbohydrate metabolic process |
| G3HMV7 | Tissue alpha-L-fucosidase | FUCA1 | 5 | 0.733 | Signalp(1.0) | carbohydrate metabolic process |

^{a}Secretion prediction according to SecretomeP server. Proteins with NN score ≧0.5 are predicted as secreted by non classical secretory pathway.
^{b}Secretion predictio [symbol] according to SignalP server. Numbers in parenthesis indicate the signal peptide probability.
^{c}Biological functions of secreted proteins were extracted from Gene Ontology an [symbol] UniprotKb. [symbol]
indicates data missing or illegible when filed

and 59 secreted growth factors for human and mouse respectively. As such, the number of identified CHO secreted growth factors constituted approximately 9-130 of the known human and mouse secreted growth factors. The other unidentified growth factors may not be expressed by CHO cells due to genetic differences between mammalian species.

### Example 5

### Validation of Secreted Growth Factors by RNA Transcript Analysis and Western Blot

### Materials and Methods

### Western Blot Validation

Proteins from day 2 supernatant samples were concentrated using membrane filtration as described above. For membrane receptor protein detection, the membrane protein fraction of CHO cells was enriched using Qproteome Cell Compartment Fractionation Kit as according to the manufacturer's instructions (Qiagen). Proteins were resolved by 10% SDS-PAGE under reducing conditions and transferred onto PVDF membranes. The membranes were blocked in 5% dry milk in TTBS (0.1% Tween-20 in TBS, w/v) and incubated overnight at 4° C. with the following antibodies: anti-LIF, 0.2 μg/ml (R&D Systems; catalog number AF-250-NA); anti-FGF8, 1:1000 (abcam; catalog number ab89550); anti-HGF, 1 μg/mL (R&D Systems; catalog number MAB294); anti-VEGF-C, 1:1000 (Angio-Proteomie; catalog number pV1006R-r); anti-BDNF, 1:1000 (abcam; catalog number ab101752); anti-GRO alpha, 1:1000 (abcam; catalog number ab9772); anti-HDGF; 1:1000 (abcam; ab43668); anti-MCSF; 1:1000 (abcam; ab9693) for secreted growth factors and Trkb antibody, 1:1000 (Cell Signal; catalog number 4606); anti-FGFR3, 1:500 (Novus Biologicals; catalog number NB100-91767); anti-FGFR4, 1:50 (abcam; catalog number ab5481); anti-CXCR2, 1:1000 (abcam; catalog number ab14935); Met antibody (6AT44), 1:100 (Novus Biologicals; catalog number NB100-25947); anti-LIFR, 1:50 (abcam; catalog number ab89792); anti-MCSF receptor, 1:1000 (abcam; catalog number ab89907) and anti-VEGF Receptor 2, 1:500 (Novus Biologicals; catalog number NB100-2382) for membrane receptors. The membranes were washed and incubated with HRP-conjugated secondary antibody diluted 1:5000 in 2% milk in TTBS for 1 h at room temperature and developed using enhanced chemiluminescence detection according to the manufacturer's instructions (Amersham Biosciences).

### Results

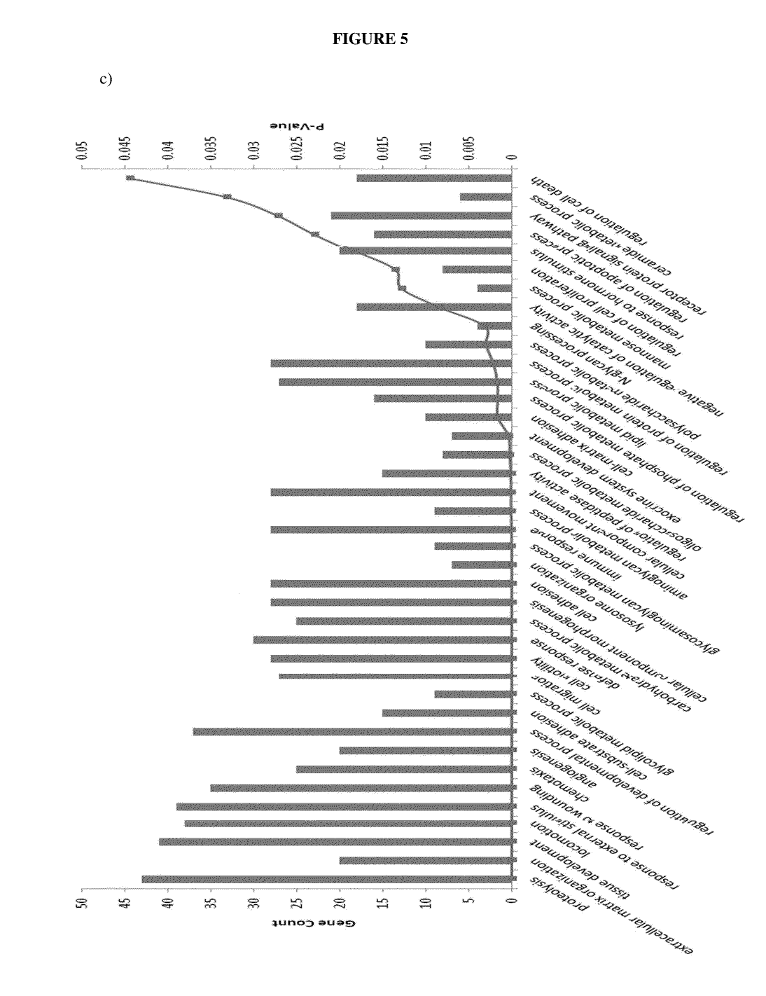

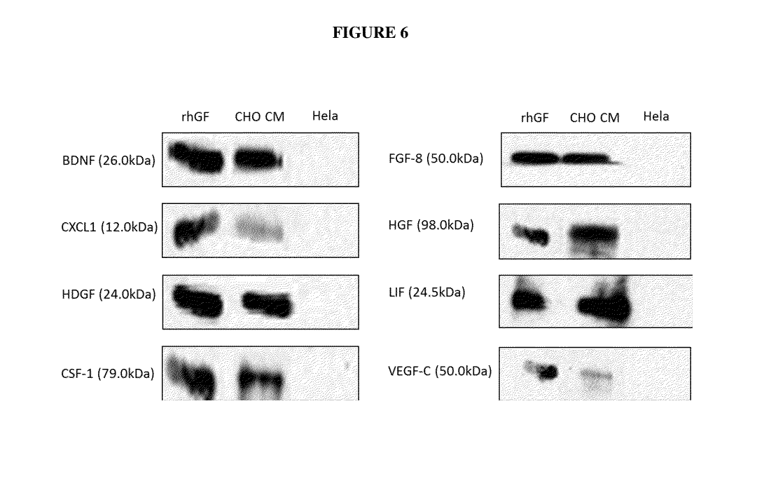

As LC-MS analysis provided evidence that growth factors were secreted in CHO-K1 supernatant, validation of their expression was performed by mining existing CHO-K1 RNAseq data and western blot analysis. The CHO-K1 transcriptome was previously sequenced and mapped by Bioprocessing Technology Institute (BTI). With reference to this in-house CHO-K1 RNAseq data, all of the identified secreted growth factors were confirmed to have mRNA transcribed in proliferating CHO-K1 cells (Table 5). To corroborate the expression of these secreted growth factors at the protein level, Western blot was performed with the same protein extract from Day 2 CHO-K1 CM as those that were used for shotgun LC-MS experiments (FIG. 6). All 8 secreted growth factors were detected by immunoblotting which confirmed their identities and eliminated the possibility of false positives. For the first time, these growth factors secreted by CHO cells were detected from experimental data and the differences between their experimental and predicted molecular weights (translated from CHO genomic sequences; UniprotKb Chinese hamster database) are tabulated in Table 6.

TABLE 5
RNA transcript analysis and western blot detection for secreted growth factors and their corresponding receptors from CHO cells

| | | | Western | | | | Western Blot^{b} | |
|---|---|---|---|---|---|---|---|---|
| | Gene | Transcript^{a} | Blot^{b} | | Gene | Transcript^{a} | | CHO |
| Ligand | Name | CHO-K1 | CHO-K1 | Membrane Receptors | Name | CHO-K1 | CHO-K1 | m250-9 |
| Brain-derived neurotrophic | Bdnf | + | + | Trkb tyrosine kinase | Trkb | − | − | − |
| factor | | | | | | | | |
| Fibroblast growth factor 8 | Fgf8 | + | + | Fibroblast growth factor receptor 3 | Fgfr3 | + | + | + |
| | | | | Fibroblast growth factor receptor 4 | Fgfr4 | | | |
| Growth regulated α protein | Cxcl1 | + | + | C-x-c chemokine receptor 1 | Cxcr1 | − | − | − |
| | | | | C-x-c chemokine receptor 2 | Cxcr2 | | | |
| Hepatocyte growth factor | Hgf | + | + | Hepatocyte growth factor receptor | Hgfr | + | + | + |
| Hepatoma-derived growth | Hdgf | + | + | — | | − | − | − |
| factor | | | | | | | | |
| Leukemia inhibitory factor | Lif | + | + | Leukemia inhibitory factor | Lifr | + | + | + |
| | | | | receptor | | | | |
| Macrophage colony- | Csf1 | + | + | Macrophage colony-stimulating | Mcsfr | − | − | − |
| stimulating factor 1 | | | | factor receptor | | | | |
| Vascular endothelial growth | Vegfc | + | + | Vascular endothelial growth factor | Vegfr2 | + | + | + |
| factor C | | | | receptor 2 | | | | |
| | | | | Vascular endothelial growth factor | Vegfr3 | − | − | − |
| | | | | receptor 3 | | | | |

^{a}In-house transcriptomic data sequenced from CHO-K1 cells by BTI
^{b}Day 2 supernatant from CHO-K1 cells was concentrated and the membrane fraction of CHO-K1 cell lysates were enriched. 20 μg of total protein for each lane was loaded onto SDS-PAGE gels. Then proteins were transferred onto PVDF and probed with indicated protein antibodies

TABLE 6
Western blot detection of secreted
growth factors from CHO-K1 cells

| | | | | | # |
|---|---|---|---|---|---|
| | | Pre- | Experi- | | Putative |
| | | dicted | mental | Differ- | N-Glyco- |
| | Gene | MW^{a} | MW^{b} | ence | sylation |
| Protein Description | Name | (kDa) | (kDa) | (kDa) | Sites^{c} |
| Brain-derived neuro- | Bdnf | 20.7 | 26.0 | 5.3 | 1 |
| trophic factor | | | | | |
| Fibroblast growth | Fgf8 | 37.9 | 50.0 | 12.1 | 1 |
| factor 8 | | | | | |
| Growth regulated-α | Cxcl1 | 10.9 | 12.0 | 1.1 | 0 |
| protein | | | | | |
| Hepatocyte growth | Hgf | 83.0 | 98.0 | 15.0 | 5 |
| factor | | | | | |
| Hepatoma-derived | Hdgf | 22.7 | 24.0 | 1.3 | 0 |
| growth factor | | | | | |
| Leukemia inhibitory | Lif | 17.4 | 24.5 | 7.1 | 6 |
| factor | | | | | |
| Macrophage colony- | Csf1 | 60.7 | 79.0 | 18.3 | 4 |
| stimulating factor 1 | | | | | |
| Vascular endothelial | Vegfc | 39.3 | 50.0 | 10.7 | 3 |
| growth factor C | | | | | |

^{a}Predicted molecular weight of proteins that are translated from CHO genomic sequences (UniprotKb database),
^{b}Observed molecular weight of proteins from CHO-K1 experimental data
^{c}Number of putative N-glycosylation sites for human homologs obtained from the UniprotKb database

Without discounting the possibility of inaccurate molecular weight readings from the Western blot results, the difference in experimental and predicted molecular weights may also be indicative of any potential protein post-translational modifications (PTMs) such as N-glycosylation or other amino acid modifications. This is because the predicted molecular weights for most of the Chinese hamster proteins in UniprotKb do not account for PTMs due to the lack of experimental data. For example, a kDa difference between experimental and predicted molecular weight was detected for the CHO hepatocyte growth factor (HGF) protein (Table 6). With reference to its human homolog (SwissProt, P14210), this protein contained 0.5 putative glycosylation sites which represents the extent of protein modification for this protein even though differences may exist between species. Hence, this information provided an indication on how heavily modified the CHO protein might be which can be invaluable for characterizing novel CHO proteins especially those without any cross-species homologs.

### Example 6

### Identification of Autocrine Growth Factors by Validating Membrane Receptors

### Materials and Methods

### Network Analysis Using MetaCore

MetaCore (GeneGo) was used to map the identified secreted growth factors into biological networks. MetaCore is an integrated software suite for functional analysis of experimental data. It is based on proprietary manually curated database of human protein-protein, protein-DNA, and protein-compound interactions. Metabolic and signaling pathways and the effects of bioactive molecules are included in the analysis. Swissprot IDs of secreted growth factors were uploaded into MetaCore platform for network analysis using the shortest path algorithm.

### Results

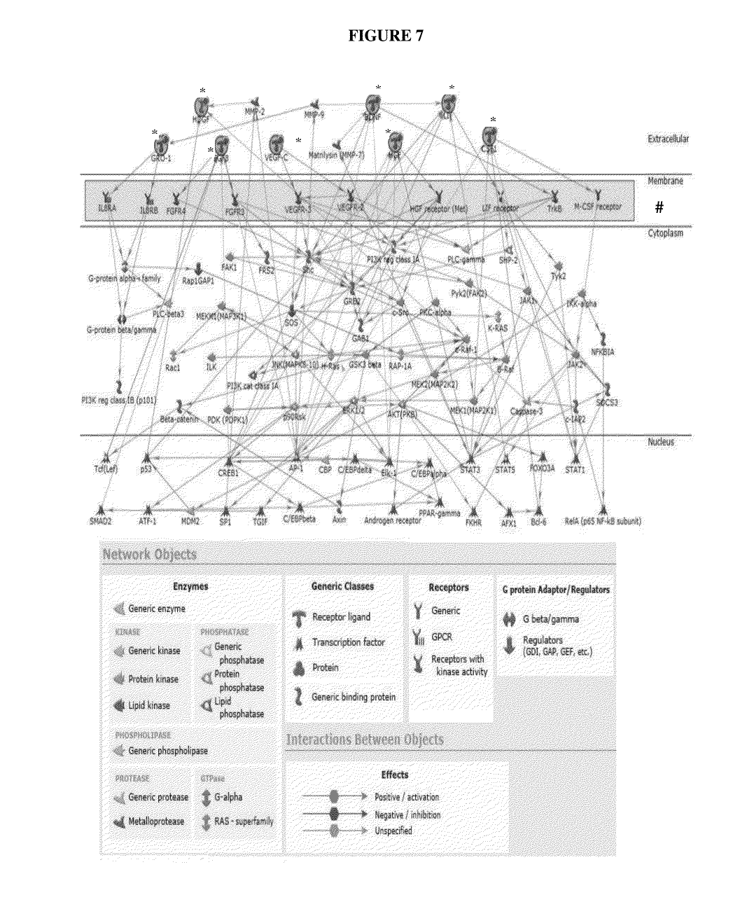

To identify autocrine growth factors from the 8 secreted growth factors, the expression of their corresponding membrane receptors were examined. The list of membrane receptor proteins were determined by analyzing their biological signaling pathways using MetaCore™ (FIG. 7) because MetaCore™ allows functional analysis of proteomics data based on high quality, manually curated literature including databases of protein information and signaling pathways (https://portal.genego.com). Network analysis indicated a total of 10 membrane receptors that corresponded to these secreted growth factors; Trkb tyrosine kinase, fibroblast growth factor receptor 3, fibroblast growth factor receptor 4, c-x-c chemokine receptor 1, c-x-c chemokine receptor 2, hepatocyte growth factor receptor, leukemia inhibitory factor receptor, macrophage colony-stimulating factor receptor, vascular endothelial growth factor receptor 2 and vascular endothelial growth factor receptor 3.

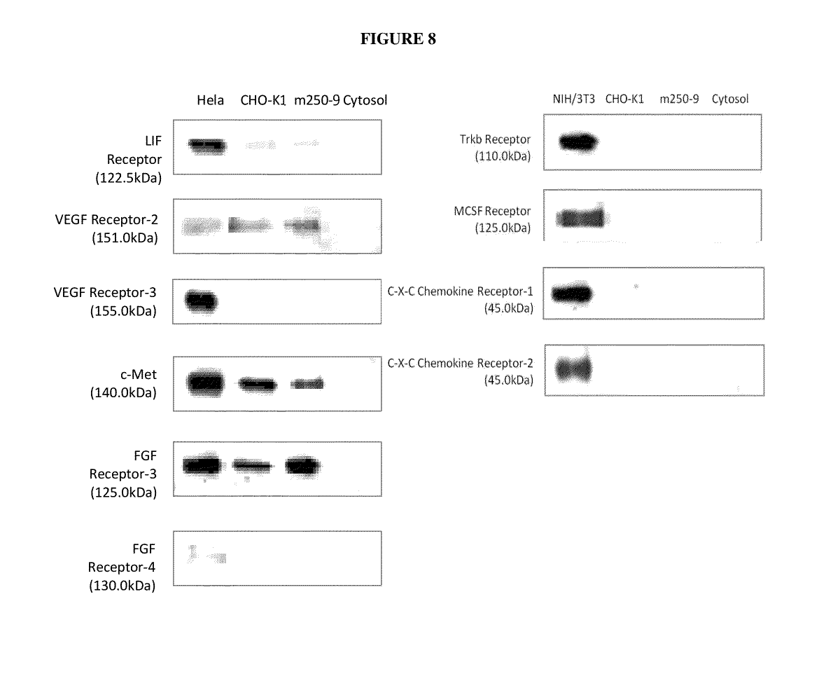

The presence of the receptor mRNA transcripts was then tested by mining existing CHO-K1 RNAseq data and the protein expression was probed by western blot of CHO cells. Out of the 10 membrane receptors identified, we detected the presence of fibroblast growth factor receptor 3 (Fgfr3), hepatocyte growth factor receptor (Hgfr), leukemia inhibitory factor receptor (Lifr) and vascular endothelial growth factor receptor 2 (Vegfr2) at the transcript and protein levels for CHO-K1 and CHO m250-9 cells (FIG. 8, Table 7). In contrast, vascular endothelial growth factor receptor 3 (Vegfr3), fibroblast growth factor receptor 4 (Fgfr4), Trkb tyrosine kinase (Trkb), macrophage colony-stimulating factor receptor (Mcsfr), c-x-c chemokine receptor 1 (cxcr1) and c-x-c chemokine receptor 2 (Cxcr2) could not be detected at the transcript and protein levels for both CHO cell-lines. The results for transcript and western blot detection of these growth factor membrane receptors are summarized in Table 5.

TABLE 7
Western blot detection of membrane receptors from CHO cells

| | | Pre- | Experi- | Differ- |
|---|---|---|---|---|
| | Gene | dicted | mental | ence |
| Protein Description | Name | MW^{a} (kDa) | MW^{b} (kDa) | (kDa) |
| Trkb tyrosine kinase | Trkb | 92.0 | — | — |
| Fibroblast growth factor | Fgfr3 | 35.7 | 125.0 | 89.3 |
| receptor 3 | | | | |
| Fibroblast growth factor | Fgfr4 | 114.9 | — | — |
| receptor 4 | | | | |
| C-x-c chemokine re- | Cxcr1 | 40.0 | — | — |
| ceptor 1 | | | | |
| C-x-c chemokine re- | Cxcr2 | 41.0 | — | — |
| ceptor 2 | | | | |
| Hepatocyte growth factor | Hgfr | 136.9 | 140.0 | 3.1 |
| receptor | | | | |
| Leukemia inhibitory | Lifr | 45.3 | 122.5 | 77.2 |
| factor receptor | | | | |
| Macrophage colony- | Mcsfr | 107.9 | — | — |
| stimulating factor | | | | |
| receptor | | | | |
| Vascular endothelial | Vegfr2 | 129.0 | 151.0 | 22.0 |
| growth factor receptor 2 | | | | |
| Vascular endothelial | Vegfr3 | 137.2 | — | — |
| growth factor receptor 3 | | | | |

^{a}Predicted molecular weight of proteins translated from CHO genomic sequences (UniprotKb database)
^{b}Observed molecular weight of proteins from CHO-K1 and CHO m250-9 experimental data

Based on the detection of membrane receptors, 4 autocrine growth factors were shortlisted such as fibroblast growth factor 8 (FGF-8), hepatocyte growth factor (HGF), leukemia inhibitory factor (LIF) and vascular endothelial growth factor-c (VEGF-C) (Table 5). The other identified secreted growth factors such as brain derived neutrothrophic factor (BDNF), growth regulated-a protein (CXCL1) and macrophage colony-stimulating factor 1 (CSF1) were not selected for in vitro functional experiments due to the lack of functional receptors to elicit an autocrine growth response. As for the hepatoma derived growth factor (HDGF) protein, its cell surface receptor is currently still unknown. This growth factor was excluded from functional studies because our objective was to identify autocrine growth factors with receptor-ligand activity.

### Example 7

### Quantification of Autocrine Growth Factors by ELISA

### Materials and Methods

### Enzyme-Linked Immunosorbent Assay (ELISA)

Secreted growth factors in Day 2, 3, 4 & 5 culture supernatant collected from CHO-K1 fed-batch bioreactor cultures were quantified using ELISA. Triplicate samples were assayed for FGF-8, HGF, VEGF-C and LIF content on ELISA plates according to the manufacturer's instructions (R&D Systems for mouse LIF and mouse. HGF; Cusabio Biotech for mouse FGF-8 and mouse VEGF-C). FGF-8, HGF, VEGF-C and LIF concentration values were obtained by linear regression of absorbance readings made with a Tecan Sunrise™ microtiter plate reader, against known concentrations of recombinant growth factors measured in serial dilution on each microtiter plate. According to the manufacturer's specifications, the ELISA assay does not cross react with other closely related growth factors or cytokines because no significant cross-reactivity was observed when various factors were tested for interference.

### Results

Four autocrine growth factors, FGF-8, HGF, VEGF-C and LIF, were shortlisted for functional studies to improve low cell density growth of CHO cells. To verify the proliferative activity of these autocrine growth factors, the highest concentration of these proteins in CM were used as starting concentrations for in vitro dose-response experiments. As the autocrine growth factors detected by LC-MS experiments were only qualitative in nature, an absolute quantification of these proteins in Day 2 (exponential phase) to Day 5 (stationary phase) CM that were sampled from CHO-K1 bioreactor cultures was performed by sandwich ELISA using 96 well plates (Table 8).

TABLE 8
ELISA detection of FGF-8, HGF, VEGF-C and
LIF in the bioreactor culture supernatant

| | FGF-8 (pg/ml) | HGF (pg/ml) | VEGF-C (pg/ml) | LIF (pg/ml) |
|---|---|---|---|---|
| Day 2 | 114 ± 16 | 54 ± 12 | 20 ± 3 | 86 ± 9 |
| Day 3 | 171 ± 10 | 129 ± 18 | 28 ± 2 | 282 ± 7 |
| Day 4 | 172 ± 9 | 179 ± 11 | 29 ± 2 | 419 ± 15 |
| Day 5 | 180 ± 10 | 173 ± 15 | 29 ± 3 | 541 ± 20 |

ELISA results indicated that the concentration for FGF-8 and LIF increased by 1.6 fold and 6.3 fold respectively from Day 2 to Day 5 CM. Likewise, the concentration for HGF and VEGF-C increased to 179±11 pg/ml and 29±2 pg/ml respectively for Day 4 CM and maintained at these concentrations on Day 5. During the onset of the stationary phase (Day 5), there was a smaller increase in growth factor concentration partly due to the slower culture growth rate and high culture media dilution during feeding. The slower growth rate of the bioreactor cultures on Day 5 (0.017±0.002 hr⁻¹) as compared to Day 2 (0.036±0.003 hr⁻¹) during the exponential phase of cell growth may have resulted in fewer growth factors being secreted by the cells. At the same period, there was also a surge in the feeding volume to cater the nutritional requirements of this high cell density culture, which essentially diluted the concentration of growth factors in the culture supernatant.

Other possibilities can also include the degradation of growth factors by proteases released or secreted by CHO cells. Many different serine proteases, cathepsins and metalloproteases (MMP) were detected from CM which may have contributed to growth factor degradation (Table 4). Cathepsins too have the ability to degrade extracellular proteins since extracellular matrix proteins have been subjected to proteolytic degradation by cathepsins in an acidic and oxidative culture environment.

Of the four autocrine growth factors, LIF provided the highest concentration on Day 5 at 541±20 pg/ml; followed by FGF-8 at 180±10 pg/ml, HGF at 173±15 pg/ml and VEGF-C at 29±3 pg/ml (Table 8). The highest concentration of growth factors was used as starting concentrations for in vitro functional studies.

### Example 8

### In Vitro Dose Response and Combinatorial Growth Experiments

### Materials and Methods

### 3-(4,5 dimethylthiazol-2-thiazyl)-2,5-diphenyl-tetrazolium bromide (MTT) Proliferation Assay

The ability of recombinant growth factors to induce cellular proliferation was evaluated by using 3-(4,5 dimethylthiazol-2-thiazyl)-2,5-diphenyl tetrazolium bromide (MTT) (ATCC) assays for static cultures. Briefly, CHO-K1 cells (50,000 cells) were plated in 96-well plates and treated with fresh media in the presence of different concentrations of recombinant growth factors. Concentrated day 2 CM was used as positive controls and no treatment was used as a negative control. After 24 hr, MTT solution (5 mg/ml) was added and the cells were incubated at 37° C. for 3 hr. A detergent was added (100 ul) to the supernatant and left at room temperature in the dark for 2 hr. Absorbance was measured at 570 nm using a Tecan Sunrise™ microplate reader (Tecan). Results were expressed as the mean±SD of results from 3 replicate wells.

### Results

To verify the functionality of the identified autocrine growth factors, culture media was supplemented with FGF-8, HGF, LIF and VEGF-C at varying concentrations to test growth stimulation for low cell density CHO-K1 cultures. The cells were seeded at 1000 cells/well (1×10⁴ cells/ml) and incubated with the growth factors in 96 well plates for 72 hr and output was measured with the MTT cell proliferation assay. No significant improvement in cell growth was observed when autocrine growth factors were tested at physiological concentrations detected from CM (0.1 ng/ml and 0.5 ng/ml). Without being bound by theory, the lack of growth stimulation by the growth factors at physiological concentrations may be due to differences of the reactive epitope between the recombinant human growth factors used in the experiment and the native CHO growth factors. Another possible contributing factor may be differences in post-translational modifications for the recombinant growth factors, such as protein folding, phosphorylation and glycosylation patterns, since they are produced using different cell types: FGF-8 and LIF is produced from E. coli, HGF is produced from Baculovirus and VEGF-C is produced from NSO cells. These factors may have resulted in lower binding affinity and signaling of the recombinant factors, and as such the lack of growth stimulation at physiological concentrations.

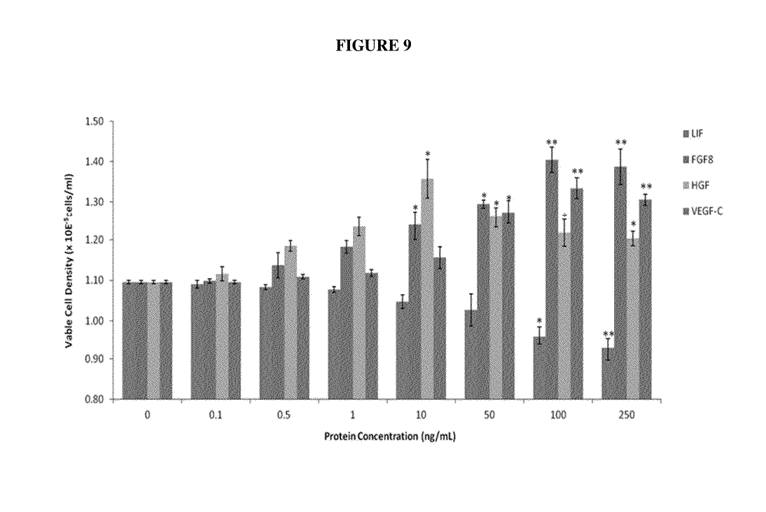

At higher protein concentrations of 1, 10, 50, 100 & 250 ng/ml, the addition of FGF-8, HGF and VEGF-C to CHO-K1 cells accelerated cell growth in a dose-dependent manner, and the optimal concentrations of growth factors for maximal growth were determined at 100 ng/ml for FGF-8, 10 ng/ml for HGF and 100 ng/ml for VEGF-C (FIG. 9). This is the first reported investigation of the mitogenic effects of HGF and VEGF-C on CHO cell growth.

In contrast, LIF inhibited CHO cell growth starting at concentrations from 10 ng/ml to a maximum concentration of 250 ng/ml (FIG. 9). The accumulation, of this growth inhibitor in CM as the culture progressed may potentially trigger cell death since the concentration of LIF was 6.3 times higher on Day 5 compared to Day 2. Given the objective of this research, only FGF-8, HGF and VEGF-C with proliferative activity were considered.

Different combinations of FGF-8, HGF and VEGF-C were tested with optimized concentrations to determine synergistic effects on CHO cell proliferation for stimulating low cell density growth. The combination of FGF-8, HGF and VEGF-C produced the best growth stimulatory effect with an improvement of 31.3% after 72 hours, compared to an improvement of 48% by CM (Table 9). Of the 3 growth factors, FGF-8 alone was sufficient to stimulate 21.4% of cell growth, followed by HGF (4.5%) and VEGF-C (2.3%). This indicated that FGF-8 is the predominant growth factor that stimulates cell proliferation in CM, with additional improvements for combinations with HGF and VEGF-C, although CM contains other growth stimulants that can further improve CHO cell growth.

TABLE 9
Combinatorial effects of recombinant FGF8,
HGF and VEGF-C on CHO-K1 cell growth

| | | | | Viable cell | |
|---|---|---|---|---|---|
| | FGF8 | HGF | VEGF-C | density^{a} | Stimula- |
| expt | (100 | (10 | (100 | (×10⁻⁵ | tion^{b} |
| no. | ng/ml) | ng/ml) | ng/ml) | cells/ml) | (%) |
| 1 | − | − | − | 1.12 ± 0.21 | — |
| (Control) | | | | | |
| 2 | − | − | + | 1.27 ± 0.15 | 13.4 |
| 3 | − | + | − | 1.29 ± 0.25 | 15.1 |
| 4 | − | + | + | 1.32 ± 0.22 | 17.9 |
| 5 | + | − | − | 1.36 ± 0.15 | 21.4 |
| 6 | + | − | + | 1.42 ± 0.22 | 26.7 |
| 7 | + | + | − | 1.44 ± 0.12 | 28.6 |
| 8 | + | + | + | 1.47 ± 0.18 | 31.3 |
| Positive | Condi- | | | 1.66 ± 0.21 | 48.2 |
| (Day 2 | tioned | | | | |
| Media) | | | | | |

^{a}Viable cell density of CHO-K1 cells (1 × 10⁴ cells/ml) cultivated in 96 well plates obtained from MTT Assay post 72 hours. Results are represented as mean of 3 replicates ± standard deviation
^{b}Percentage improvement of viable cell density relative to the control culture condition

### Example 9

### Effect of Growth Factor-Neutralizing Antibodies on CHO Cell Growth

### Materials and Methods

### Effect of Neutralizing Antibodies on Growth of CHO Cells.

CHO-K1 cells were grown at a cell density of 1×10⁴ cells/ml in fresh media supplemented with indicated concentrations of neutralizing antibodies and isotype-specific control antibodies for 72 hours in 96 well plates. The list of neutralizing antibodies used in this experiment along with their respective formulations is tabulated in Table 10. Cell growth was measured using the MTT cell proliferation assay.

TABLE 10
List of neutralizing antibodies used
for growth factor functional studies

| | | Catalog | Preserva- |
|---|---|---|---|
| Antibody Description | Company | Number | tives |
| mouse IgG1 isotype control | RND Systems | MAB002 | 5% |
| antibody | | | Trehalose |
| mouse monoclonal anti- | abcam | ab89550 | None |
| FGF8 antibody | | | |
| mouse monoclonal anti-HGF | RND Systems | MAB294 | 5% |
| antibody | | | Trehalose |
| rabbit polyclonal anti-VEGF- | angio- | pV1006R-r | None |
| C antibody | proteomie | | |

### Results

Since it was demonstrated that concentrated Day 2 CM improved CHO-K1 cell growth, the possibility that FGF-8, HGF and VEGF-C in CM were responsible for this effect was investigated. Hence, concentrated CHO cell CM was treated with neutralizing antibodies to verify the presence of autocrine growth factors in CM. Mouse IgG immunoglobulin was used as an isotype control for the neutralizing antibodies to ensure that growth inhibition was not affected by other variable factors such as differences in antibody isotypes or from added preservatives to the antibody formulation. Even though most of the antibodies did not contain any preservatives, the ones that did, contained 5% Trehalose, a non-toxic disaccharide sugar typically used to prevent protein hydrolysis at acidic and high temperatures.

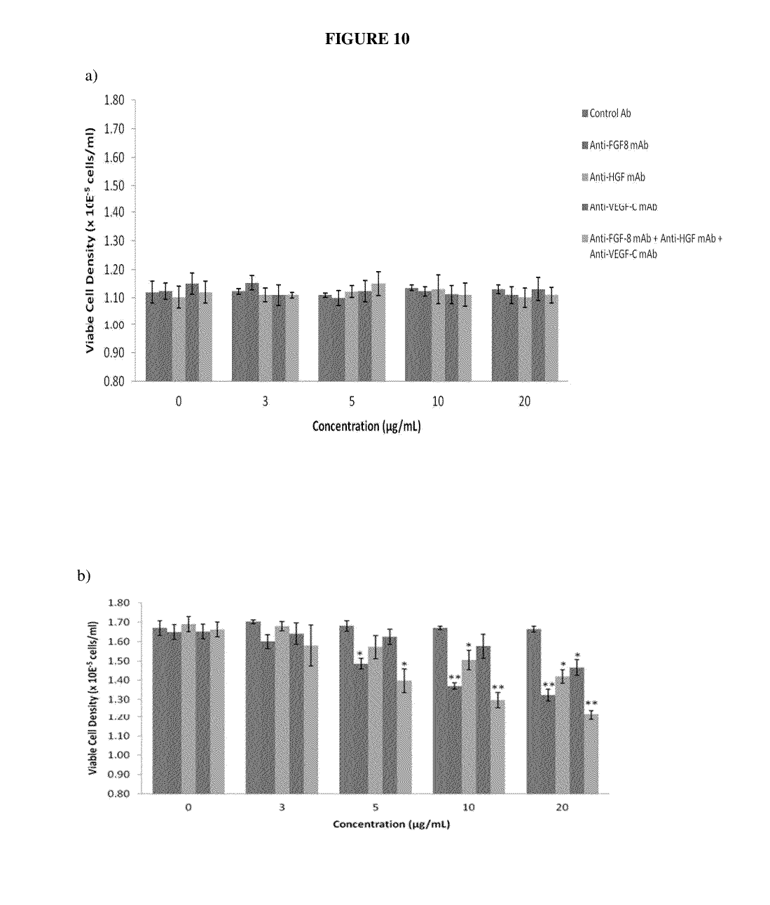

Stimulation of growth was inhibited when CM was treated with increasing concentrations of anti-FGF-8 mAb, anti-HGF mAb or anti-VEGF-C mAb respectively (FIG. 10b). The extent of neutralization by each of these antibodies reflected an increasing trend of proliferative activity by these autocrine growth factors in the following order; VEGF-C, HGF and FGF-8. Furthermore, neutralization of these autocrine growth factors was consistent with their order of growth stimulation when recombinant proteins were tested in vitro (Table 9). As such, treatment with all 3 neutralizing antibodies resulted in the least improvement of cell growth compared to other conditions. In contrast, there was no difference in growth when the cells were cultured in fresh media treated with these neutralizing antibodies (FIG. 10a). These results concluded that growth stimulation by FGF-8, HGF and VEGF-C was negated with the treatment of neutralizing antibodies and that these growth factors are the main growth stimulants in these concentrated CM, corroborating with the results shown in Table 9.

### Example 10

### Extracellular Signal-Regulated Kinase (ERK) Pathway Activation by Autocrine Growth Factors

### Materials and Methods

### Phosphorylation Studies

Western blot analysis on protein phosphorylation of ERK1/2 was performed with the addition of these recombinant growth factors to 60 ml, CHO-K1 shakeflask cultures with cells harvested at 0 min, 1 min, 5 min, 15 min and 30 min. 100 ng/ml of FGF-8, 10 ng/ml of HGF and 100 ng/ml of VEGF-C were either added individually or as a combination to, investigate their roles in activating the ERK signaling pathway. ERK phosphorylation was probed on the Thr202/Tyr204 site and total ERK2 expression was used as a loading control. The primary antibodies used for phosphorylation studies consist of:—Phospho-p44/42 MAPK (Erk1/2) (Thr202/Tyr204) (D13.14.4E) XP® Rabbit mAb; 1:1000 (Cell Signaling; catalog number 4370P) and p44/42 MAPK (Erk1/2) (137F5) Rabbit mAb (Cell Signaling; catalog number 4695).

### Results

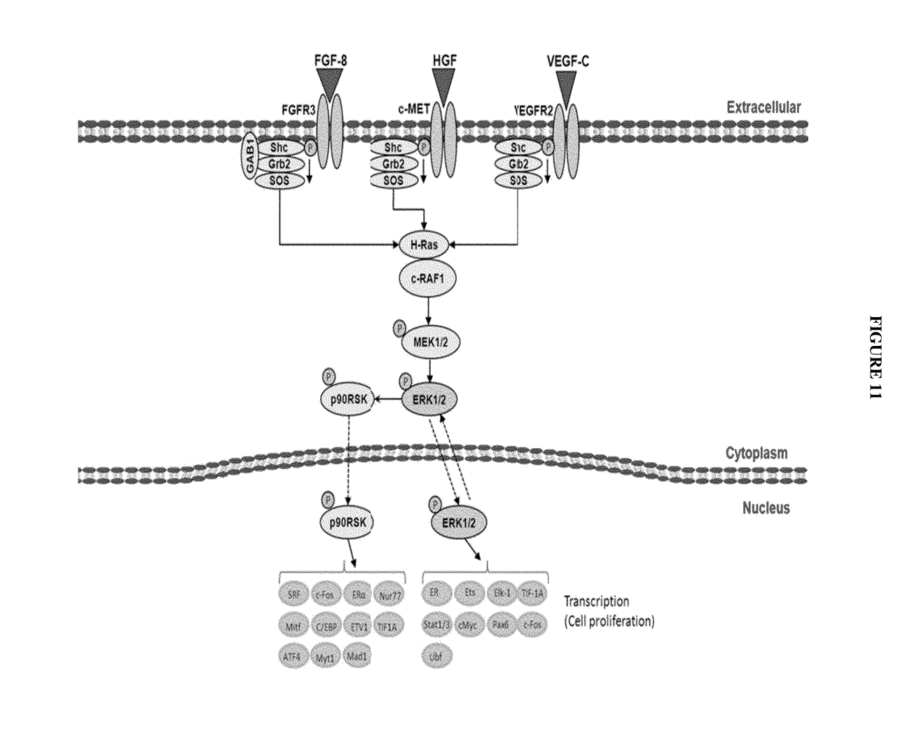

FGF-8, HGF and VEGF-C were mapped on a network converging on the ERK pathway (FIG. 11), a mitogen-activated protein kinase (MAPK) cascade involved in regulation of cell proliferation in mammalian cells. FGF-8, HGF and VEGF-C stimulate proliferation of many cell types including cancer cell-lines by binding and activating cell-surface receptors with intrinsic protein kinase activity. The stimulation of tyrosine kinase receptors like FGF receptor 3, HGF receptor and VEGR receptor 2 provokes the activation of MAPKs in the signaling pathway by activating ERKs to translocate to the nucleus and transactivate transcription factors, modulating gene expression to promote growth and mitosis.

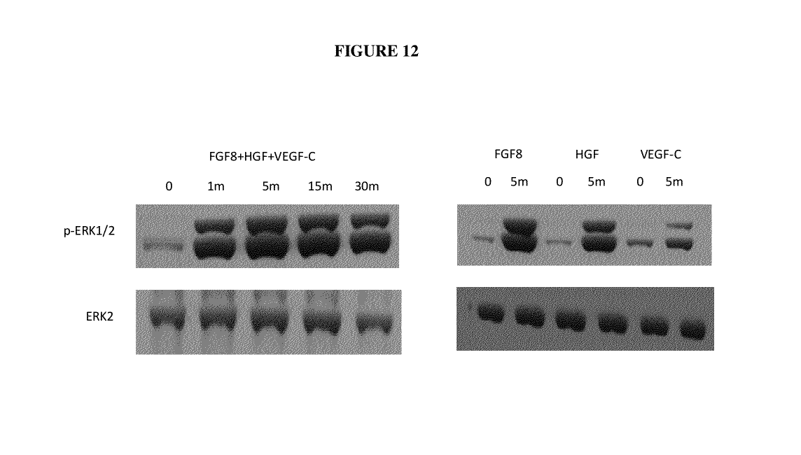

To validate that CHO cell growth is stimulated by FGF-8, HGF and VEGF-C, a Western-blot analysis on protein phosphorylation of ERK1/2 was performed with the addition of these recombinant growth factors to CHO-K1 shakeflask cultures Results indicated that after 1 min, FGF-8, HGF and VEGF-C phosphorylated ERK1/2 when they were added as a combination (FIG. 12). In addition, it was observed that the band intensity at 5 min was at its saturation point for this combination of growth factors.

When these growth factors were added individually, the greatest intensity of ERK1/2 phosphorylation was achieved by FGF-8, followed by HGF and VEGF-C respectively. This suggests that the potency of these growth factors may depend on its ability to activate ERK1/2 for growth stimulation because ERK1/2 phosphorylation corresponded to the differences in growth stimulation by these growth factors when cells were cultured in vitro (Table 9). Nonetheless, other pathways should also be stimulated by these growth factors to result in the complementary effects of increasing growth stimulation when these growth factors are used in combinations (Table 9).

### Example 11

### FGF-8, HGF and VEGF-C Promoted Single Cell Growth of CHO Cell-Lines

### Materials and Methods

### Single Cell Cloning Experiments

CHO-K1 cells were diluted in a protein free medium. CHO DG44 cells were grown in either Hyclone HyQ® PF-CHO™ (Thermo Scientific) or Hyclone HyQ® PF-CHO™: CD-CHO PF media (Life Technologies) (1:1) with added hypoxanthine, and thymidine from a 100×HT solution (Life Technologies). Experiments were conducted with 4 conditions: (1) culture media alone, (2) culture media with added recombinant growth factors consisting of 100 ng/ml of human FGF-8 (Life Technologies), 10 ng/ml of human HGF (RND Systems) and 100 ng/ml of human VEGF-C(RND Systems), (3) culture media with 10 fold concentrated CHO-K1 day 2 CM supplemented at 10% (v/v), and (4) culture media supplemented with 100 (v/v) FBS (Life Technologies). To study the complementary effects of autocrine and paracrine growth factors on single cell growth, basal media with 2 g/l of recombinant human albumin (Novozymes Biopharma), 10 ng/ml of recombinant human epidermal growth factor (Life Technologies), 10 μg/ml of recombinant human insulin (SAFC Biosciences) and 100 ng/ml of recombinant human IGF-1 (Life Technologies) was used for comparison. Cells were seeded at 1 cell per well for a total of 30 wells in 96-well plate (Corning) in volumes of 100 μl. Each well was checked microscopically for single cells. Wells with >2 cells were eliminated and additional wells were seeded. Cells were incubated for up to 28 days at 37° C. in 5% CO2 and 70% humidified air, under static conditions and the outer wells of each plate were filled with PBS to avoid evaporation of media. For all experiments, plates were screened for colony growth by microscopic verification. Wells with greater than 40% confluence at 28 days post seeding were scored as positive. Reported values are average±standard deviation from duplicate experiments.

### Results

To determine if recombinant growth factors such as FGF-8, HGF and VEGF-C were able to improve the growth of single CHO cells, CHO cells were diluted to a concentration of 1 cell/well in 96 well plates, and added basal media supplemented with growth factors to each well. Each media, with and without supplements, were tested using 30 single cell clone wells and their cloning efficiencies were compared after 28 days post seeding. Two independent suspension CHO cell-lines previously adapted in protein-free medium, CHO-K1 and CHO m250-9 (a CHO-DG44 variant expressing a human anti-rhesus D mAb) were used.

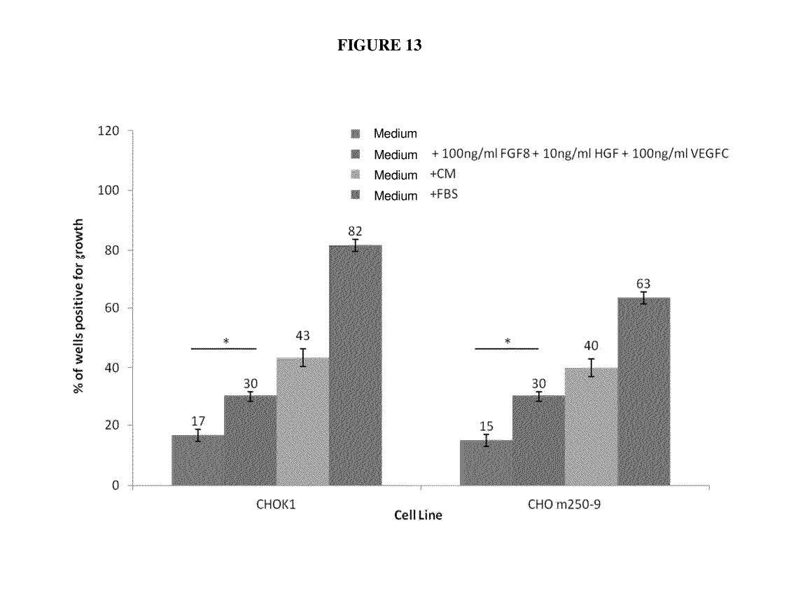

For both CHO-K1 and CHO m250-9 cells, culture media supplemented with the combination of FGF-8, HGF and VEGF-C provided a cloning efficiency of up to 30%, which is a 2-fold improvement compared to unsupplemented media (FIG. 13). Addition of CM improved the cloning efficiency to 43% and 40% for CHO-K1 and CHO m250-9 cells respectively, higher than that achieved by media supplemented with 100 ng/ml FGF-8, 10 ng/ml HGF and 100 ng/ml VEGF-C. FBS supplemented media yielded even higher cloning efficiencies because it is rich in nutrients and growth factors.

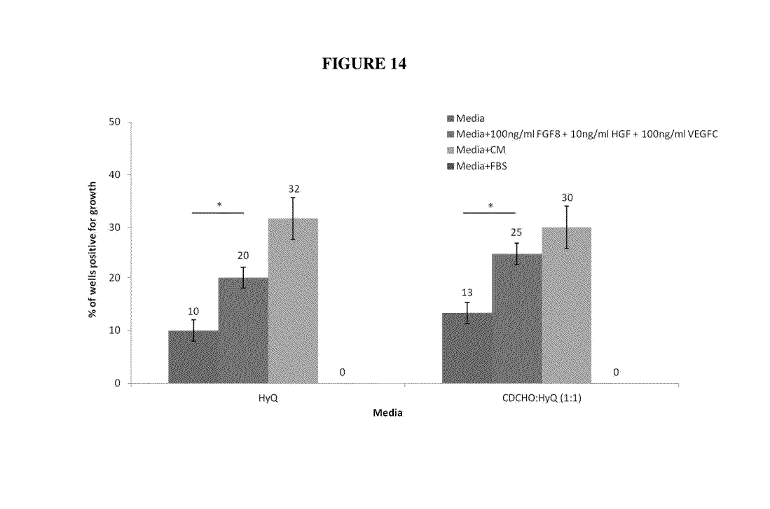

To alleviate concerns with regards to cell line peculiarity of CHO cell lines previously adapted to protein-free medium shown in FIG. 13, the same experiment was performed on a CHO-DG44 host cell line cultivated in commercial media. For these CHO-DG44 cells, three combinations of basal media were tested using commercially available protein-free media; CD-CHO™ media, HyQ PF CHO media and CD-CHO™: HyQ PF CHO media (1:1). The results indicated that single CHO-DG44 cells failed to grow in CD-CHO media alone but could grow in HyQ PF CHO and CD-CHO™:HyQ PF CHO media (1:1) with cloning efficiencies of 10% and 13% respectively (FIG. 14). While it is possible that high concentrations of glucose or glutamine in culture media can lead to increased metabolism by CHO cells and result in inhibitory levels of lactate and ammonia, it is unlikely for CD-CHO™ media to have this inhibitory effect on single cell growth because the metabolism of single cells is insufficient to generate these inhibitory levels of lactate and ammonia. It is therefore more likely that CD-CHO™ media may have missing nutrients that are essential for single CHO-DG44 cell growth. Since the data indicated that single. CHO-DG44 cells grew when CD-CHO™ media was diluted using HyQ PF CHO media, nutrients may have been supplemented by HyQ PF CHO media since CHO-DG44 cells could grow in HyQ PF CHO media alone. Unfortunately, the differences in both media compositions could not be evaluated since they are proprietary formulations.

To determine the cloning efficiencies of identified autocrine growth factors on CHO-DG44 cells, FGF-8, HGF and VEGF-C were added to HyQ PF CHO and CD-CHO™: HyQ PF CHO basal media (1:1) (FIG. 14). Results indicated that basal media supplemented with 100 ng/ml FGF-8, 10 ng/ml HGF and 100 ng/ml VEGF-C improved single cell growth with cloning efficiencies of 20% and 25% when these recombinant growth factors were added to HyQ PF CHO and CD-CHO™: HyQ PF CHO (1:1) respectively. Similar to the CHO-K1 and CHO m250-9 cell-lines, the cloning efficiencies achieved by the added growth factors were approximately 2-fold higher as compared to un-supplemented basal media, and cloning efficiencies for media with added CM were higher than those with added FGF-8, HGF and VEGF-C. It was also interesting to note that supplementation with FBS resulted in no single cell growth for this cell line, in contrast to the results in FIG. 13. Since CHO-DG44 single cell cloning has been successful with the use of medium supplemented with HT and FBS, without being bound by theory, it is possible that this observation may be due to conflicting signals being stimulated by these particular media and FBS. Nonetheless, this observation highlights the merits of a growth factor supplementation strategy to improve single cell cloning, since this will likely work with most CHO cell-lines and culture media. This also demonstrated that the autocrine growth factors can be applied for the single cell cloning of another CHO cell line cultivated in different protein free media.

### Example 12

### Complementary Effects of Autocrine and Paracrine Growth Factors for Single Cell Growth

### Materials and Methods

### Cell Culture

CHO cells were diluted to 1 cell/well and seeded into 96 well plates. Basal medium was used to culture CHO-K1 cells and CDCHO™: HyQ (at 1:1) with HT for CHO-DG44 cells. Seven conditions were tested which included media alone; media+(A; 100 ng/mL FGF-8+10 ng/mL HGF+100 ng/mL VEGF-C); media+(B; 100 ng/mL IGF-1+10 μg/mL insulin+10 ng/mL EGF+2 g/L human albumin); media+A+B; media+conditioned media (CM); media+10% FBS; and media+10% FBS+A. For each condition, a total of 30 replicates were used and wells with greater than 40% confluence at 28 days post seeding were scored as positive. The percentage of positive wells is indicated for both CHO-K1 and CHO-DG44 cell lines.

### Results

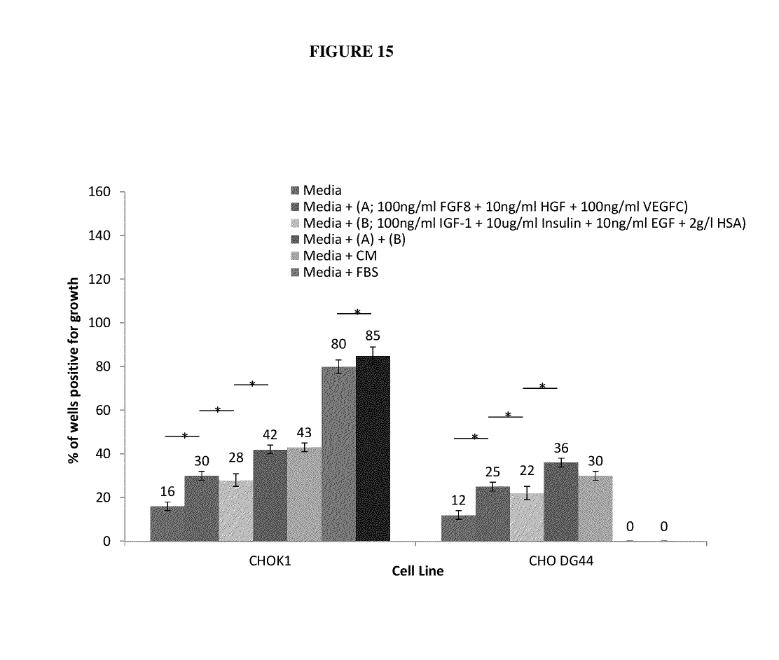

To further improve the cloning efficiencies of this cloning media, combinations with other paracrine growth factors that stimulate alternative proliferative pathways may be required. Hence, the effects of supplementing both autocrine and paracrine growth factors to basal media for CHO-K1 and CHO-DG44 cells was investigated (FIG. 15) using a combination of paracrine growth factors, IGF-1, insulin, EGF and human albumin (HA). The paracrine growth factor combination achieved a cloning efficiency of 28% and 22% for CHO-K1 and CHO-DG44 cells respectively, similar to that achieved by the autocrine growth factors. Combinations of these 2 sets of growth factors yielded cloning efficiencies of 42% and 36% for CHO-K1 cells and CHO-DG44 cells respectively. These cloning efficiencies demonstrated an improvement of up to 3-folds compared to un-supplemented media and were better than those achieved by media supplemented with autocrine growth factors (25-30%) or paracrine growth factors (22-28%) alone, while being comparable to that when CM is used. Without being bound by theory, it is possible that the higher cloning efficiencies obtained were due to the complementary effects of various signaling pathways and growth mechanisms by these growth factors.

In addition to the ERK pathway, other signaling pathways may have been activated by these paracrine growth factors. Furthermore, it also functions as a ligand/lipid carrier and an antioxidant agent for cell growth.

When FBS was used as a cloning media supplement, up to 80% of CHO-K1 single cells grew to confluence, and there was an additional 5% improvement in the cloning efficiency when autocrine growth factors were supplemented to FBS containing media (FIG. 15). This suggests that different pathways for cell proliferation may be activated by the autocrine growth factors and FBS, and that caused a complementary effect to further improve cloning efficiency. On the other hand, it was interesting to observe that CHO-DG44 single cells could not survive in FBS supplemented media similar to the results shown in FIG. 14.

Without being bound by theory, the higher cloning efficiencies obtained may due to the culmination of various signaling pathways and growth mechanisms by these growth factors. In addition to the ERK pathway, other growth signaling pathways may have been activated by the paracrine growth factors.

### CONCLUSION

The growth of CHO cells was compromised when CHO cells were seeded at low cell densities. Upon addition of CM, initial growth rate was improved which suggested that autocrine growth factors secreted in CM by CHO cells promoted cell growth even at low cell densities. The use of complementary shotgun proteomic approaches to, discover low abundant secreted growth factors in CHO-K1 fed-batch bioreactor cultures has been demonstrated. As such, the combination of complementary tandem mass spectrometry approaches and cross-species database searches enabled the discovery of 290 secreted proteins, 8 of which were growth factors. While most of these secreted proteins were unraveled by VO, peptide discovery unique to SG2 also improved the peptide coverage of VO identified proteins. The expression of these secreted growth factors was validated by mining RNAseq data and confirmed by western blot. Autocrine growth factors were then shortlisted by identifying the expression of their corresponding membrane receptors in CHO cells. This led to the shortlisting of 4 protein ligands, FGF-8, HGF, VEGF-C and LIF, for in vitro functional studies. These growth factors were quantified in CM by ELISA to determine starting concentrations for dose response experiments. FGF-8, HGF and VEGF-C each promoted CHO cell growth at low seeding densities whereas LIF inhibited cell growth. In addition, the combination of FGF-8, HGF and VEGF-C demonstrated synergistic effects for growth stimulation by activating the ERK pathway. To our knowledge, this is the first known example of autocrine growth factors used to improve CHO cell proliferation with the targets identified from CHO cell CM. Finally, the proliferative activity of shortlisted autocrine growth factors was tested and these growth factors were supplemented to chemically defined media to develop a serum-free and fully defined single cell cloning media for CHO cells. By supplementing identified autocrine growth factors to protein-free chemically defined media, the combination of FGF-8, HGF and VEGF-C improved single cell cloning efficiencies of CHO cells. In addition, complementary effects of these autocrine growth factors with other paracrine growth factors was demonstrated, to yield higher cloning efficiencies for the development of a single cell cloning media that is serum-free and fully defined.

### APPLICATIONS

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of culturing a cell comprising the step of contacting said cell with a cell culture medium comprising at least one of:
a) FGF
b) VEGF, or
c) HGF.

2. The method as claimed in claim 1, wherein the cell is a mammalian cell.

3. (canceled)

4. The method as claimed in claim 1, wherein the cell is selected from a CHO, MDCK, HEK, HeLa, A549, COS, Hep-G2, hybridoma, NSO, BHK, Vero and PerC6 or MCF-7 cell.

5. The method as claimed in claim 4, wherein the cell is a CHO cell.

6.-7. (canceled)

8. The method as claimed in claim 5, wherein the CHO cell is a single cell clone.

9. The method as claimed in claim 1, wherein the culture medium comprises FGF, VEGF, and HGF.

10. The method as claimed in claim 9, wherein the culture medium comprises FGF-8, VEGF-c, and HGF.

11. The method of claim 10, wherein the FGF-8, VEGF-c and HGF are each provided at a concentration of about 0.02 ng/ml to 250 ng/ml.

12. The method of claim 11, wherein the FGF-8, VEGF-c and HGF are each provided at a concentration of about 10 ng/ml to 100 ng/ml.

13. The method of claim 11, wherein the FGF-8 is at a concentration of about 100 ng/ml, the VEGF-c is at a concentration of about 100 ng/ml, and the HGF is at a concentration of about 10 ng/ml.

14.-15. (canceled)

16. The method of claim 1, wherein the culture medium is a serum-free medium.

17. The method of claim 16, wherein the culture medium is a serum-free medium supplemented with IGF, Insulin, EGF and HSA.

18. The method as claimed in claim 1, wherein the cell is cultured for about 14 to about 28 days at about 30° C. to about 37° C. in about 0% to about 8% CO₂.

19. The method as claimed in claim 1, wherein said method results in an increase in the cloning efficiency of the cultured cell by about 10% to about 26% relative to a cloned cell cultured in a cell culture medium that is not supplemented with at least one of FGF, VEGF, or HGF.

20. A cell culture medium for culturing a cell comprising at least one of FGF, VEGF, or HGF.

21. The cell culture medium as claimed in claim 20, wherein the medium comprises:
a. FGF-8,
b. VEGF-c, and
c. HGF.

22. The cell culture medium of claim 21, wherein the FGF-8, VEGF-c and HGF are each provided at a concentration of about 0.02 ng/ml to 250 ng/ml.

23. The cell culture medium of claim 22, wherein the FGF-8, VEGF-c and HGF are each provided at a concentration of about 10 ng/ml to 100 ng/ml.

24. The cell culture medium of claim 22, wherein the FGF-8 is at a concentration of about 100 ng/ml, the VEGF-c is at a concentration of about 100 ng/ml, and the HGF is at a concentration of about 10 ng/ml.

25.-26. (canceled)

27. The cell culture medium of claim 20, wherein the cell culture medium is a serum free medium.

28. The cell culture medium of claim 27, wherein the cell culture medium is a serum free basal medium.

29.-30. (canceled)

31. A cell produced by the method of claim 1.

32.-33. (canceled)
